(19) United States
(12) Patent Application Publication
(10) Pub. No.: US 2008/0227978 A1
(43) Pub. Date: Sep. 18, 2008

(54) Method for the Preparation of Enantiomerically Enriched Secondary Alcohols by the Addition of Organoaluminium Reagents to Carbonyl Compounds

(75) Inventor: Simon Woodward, Nottingham (GB)

Correspondence Address: IPLM GROUP, P.A., POST OFFICE BOX 18455, MINNEAPOLIS, MN, 55418, US

(73) Assignee: The University of Nottingham

(21) Appl. No.: 11/814,983

(22) PCT Filed: Jan. 26, 2006
(86) PCT No.: PCT/GB2006/000271

§ 371 Date: May 13, 2008

Related U.S. Application Data
(60) Provisional application No. 60/775,974, filed on Sep. 28, 2005.

(30) Foreign Application Priority Data
Jan. 27, 2005 (GB) 0501699.3

(51) Int. Cl. C07F 5/06; C07C 29/136; C07F 9/6584; C07F 9/6568; C07C 319/02; C07C 305/02; C07C 309/63; C07C 39/14; C07C 211/45; C07F 9/06

(52) U.S. Cl. 544/225; 568/876; 568/12; 568/62; 558/31; 558/46; 558/51; 568/735; 564/307; 564/12

(57) ABSTRACT

A process for converting a carbonyl group within a substrate to a chiral alcohol moiety comprising reacting the carbonyl containing substrate with an organoaluminium reagent in the presence of a Group 5-12 transition metal based catalyst which is complexed with a chiral ligand; novel chiral ligands and organoluminium reagents, a chiral ligand or organoaluminium reagent for use in the process.

### BACKGROUND OF THE INVENTION

1. Field of the Invention

The present invention relates to a process involving the catalysed enantioselective addition of various organoaluminium reagents (and other related reagents) to prochiral carbonyl compounds and specifically to the enantioselective production of chiral secondary alcohols through transition metal-catalysed additions of organoaluminium (and other related) reagents to aldehydes. The invention comprises: (A) an organoaluminium (or related) reagent, which may be formulated to give improved reactivity and/or convenience of handling; (B) a chiral ligand that is utilised in conjunction with a transition metal and chosen to control specificity, composition and yield of the desired product(s); and (C) the transition metal source and associated reaction conditions.

2. Description of the Prior Art

General. A number of routes exist to the preparation of chiral secondary alcohols sec-R¹R²CHOH (R¹, R²=alkyl, alkenyl, alkynyl, aryl allyl, heteroaryl and appropriate substituted variants thereof) of high enantiomeric purity which involve the modification of prochiral aldehydes (R¹CHO) or ketones (R¹C=OR²). Fujisawa has disclosed a catalytic process of relevance to the present invention [T. Ichiyanagi, S. Kuniyama, M. Shimizu, T. Fujisawa, Chem. Lett. 1998, 1033-1034]. Under Ni(acac)₂ catalysis (0.1-10mol %), AlR₃ (R=Me, Et, Bu¹) species were added, in high efficiency, to aryl, alkenyl and alkyl aldehydes under phosphane (PPh₃, PBu₃, P(OR)₃ (R=Et, Ph), diphenylphosphinoethane, diphenylphosphinobutane) promotion. Importantly, no potential for a catalytic asymmetric process was investigated or disclosed in this publication.

### SUMMARY OF THE INVENTION

The invention relates to a process for converting a carbonyl group within a substrate to a chiral alcohol moiety comprising reacting the carbonyl containing substrate with an organoaluminium reagent in the presence of a Group 5-12 transition metal based catalyst which is complexed with a chiral ligand.

Preferably the process is for the preparation of enantiomerically enriched secondary alcohols of formula sec-R¹R²CR³OH (where R¹, R²=alkyl, alkenyl, alkynyl, allyl, aryl, heteroaryl and appropriate substituted variants thereof) through transition metal-catalysed addition of organoaluminium reagents.

Preferably therefore the carbonyl containing substrate is of formula SC

(SC) R¹R²CO

and the chiral alcohol is of formula PA

(PA) R¹R²R³COH

wherein R¹, R² and R³are each independently selected from C₁-₂₄ alkyl, alkenyl, allyl, alkynyl, aryl, heteroaryl each of which may be substituted, or one of R¹ and R² is H.

The process has three aspects: (A) an organoaluminium reagent; (B) a chiral ligand that is utilised in conjunction with a transition metal and chosen to control specificity, composition and yield of the desired product(s); and (C) a transition metal source. Racemic presentation hereinafter implies the presence of any desired stereoisomer or a racemic mixture thereof. All three of these aspects include novel concepts which have not been reported before. The combination of these three aspects to afford asymmetric processes for the production of secondary alcohols is without any prior precedence.

The present invention defines very significant improved catalytic activities (TOF>50-400 h⁻¹ possible) at low catalyst loadings.

Optionally when the carbonyl containing compound is an aldehyde, the transition metal is Ni and the organometallic is AlR³R⁴R⁵ or is complexed with a polyamine (1)

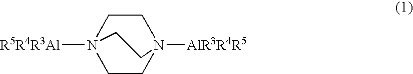

where R³,R⁴ and R⁵ are all methyl or are all ethyl, the chiral ligand is not L1

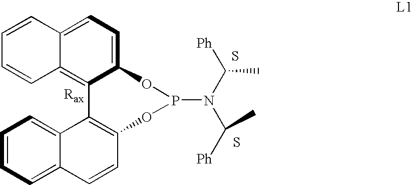

The organoaluminium reagent may be complexed with an amine or polyamine (hereinafter (poly)amine) capable of acting as a Lewis base in binding to the organoaluminium reagent. An organoaluminium—(poly)amine complex suitably comprises aluminum to (poly)amine as defined in a ratio selected from 1:1 to 1:4, preferably 1:1, 1:2 or 1:4, most preferably 1:2.

Suitably the organoaluminium reagent has the general structure AlR³R⁴R⁵ wherein each R³ to R⁵ is independently selected from C₁₋₂₄ alkyl, alkenyl, alkynyl, alkoxy, aminoalkyl, aryl, heteroaryl, allyl and substituted versions thereof, wherein substituents are selected from alkyl, alkoxy, amino, alkenyl, alkynyl, allyl, or one or two of R⁴ and R⁵ are halide, preferably chlorine or bromine or pseudohalide, or R⁵ is H, and wherein when R⁵ is H, the organoaluminium reagent may be associated with one or two additional molecules of AlR³R⁴R⁵, R⁵ being H, in dimerised form. We have found that that hydride has a tendency to spontaneously dimerise.

Fujisawa, hereinbefore referred; disclosed a non polyamine-coordinated organoaluminium reagent in a racemic process as hereinbefore described. We have surprisingly found that the process of the invention provides for ease of handling, the polyamine coordinated organoaluminium reagent (1) is a solid phase reagent which provides more convenient and safer handling than the liquid phase uncoordinated organoaluminium reagent, which is unprecedented.

The organoaluminium—polyamine complex is preferably of the formula (1a):

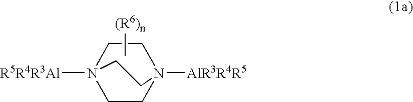

wherein each R³ to R⁵ are as hereinbefore defined and one or more R⁶ are as hereinbefore defined for R³ and n is zero or is a whole number integer selected from 1 to 12, or is of the formula (1b):

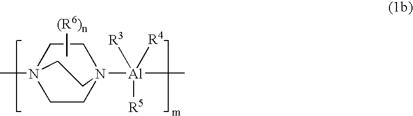

wherein each R³, R⁴, R⁵ is as hereinbefore defined and m is a whole number integer selected from 2 to 300.

Preferably R³ and R⁴ are selected from C₁₋₅ alkyl, C₃₋₁₄ alkenyl, alkynyl, allyl, aryl, preferably C₁₋₅ alkyl, C₃₋₁₄ alkenyl, and R⁵ is selected from H, C₁₋₅ alkyl, C₃₋₁₄ alkenyl, alkynyl, allyl, aryl and halide, preferably H, C₃₋₁₄ alkenyl, alkynyl and allyl.

Preferably in an organoaluminium reagent of formula (1), (1a) or (1b), R³ is different to and of different reactivity to at least one of R⁴ and R⁵ such as to induce addition of R³ to the carbonyl containing substrate.

Preferably the organoaluminium—polyamine complex is of the formulae (2) to (5) or (14) to (17).

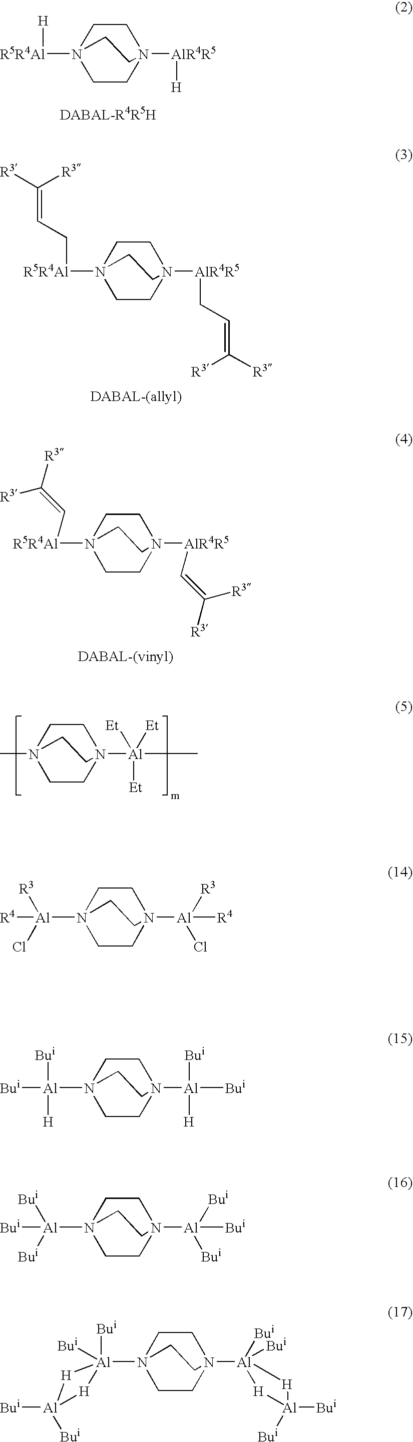

wherein each R³′ or R³″ independently, where shown, is as hereinbefore defined for R³ or one of R³′ and R³″ is H, and m is from 2 to 50.

In a particular advantage the organoaluminium—polyamine complex is a solid and is used in coated, pelletized or tabletised form, wherein a coating may be selected from a binding additive such as polyacrylate.

The organoaluminium reagent (A). In one aspect, the present invention is directed to the preparation of amine-coordinated organoaluminium species which may be formulated to give improved reactivity or convenience of handling. Direct reaction of electron rich amines NR⁶₃ with organoaluminium species: AlR³R⁴R⁵ as hereinbefore defined affords Lewis acid-Lewis base adducts whose reactivity (including in catalytic reactions) is often different to that observed with the parent organoaluminium reagent. Additional benefits are also realised and these include: reduction of the pyrophoric nature of the parent organoaluminium species, improved hydrolytic stability and modification of reactivity subsequent to the primary catalytic reaction. In the preferred embodiment of this aspect a poly amine, such as DABCO (1,4-diazabicyclo(2.2.2)octane) is used to form the DABAL reagents as exemplified by structures (1)-(4) above. In this way the mass contribution of the amine partner is minimised. The organoaluminium reagent may be reacted with the amine directly or may be initially modified (e.g. AlHBu¹₂ can be used to hydroaluminate alkenes, allenes (e.g. leading to 3, R⁴,R⁵=Bu¹) or alkynes (e.g. 4 R³″=H) prior to addition of DABCO. Similarly, AlMe₃/Cp₂ZrCl₂ may be used to afford carboaluminated alkynes [E. Negishi, N. Okukado, A. O. King, D. E. van Horn, B. I. Spiegel, J. Am. Chem. Soc. 1978, 100, 2254-2256] prior to DABCO complexation (affording 4 R³″=Me).

The DABAL reagents can be handled as solutions, as oils, or colourless crystalline solids. The nature of the products can be altered by changing the ratio of DABCO to organoaluminium reagent. In this aspect the physical properties of the derived DABAL reagents are significantly changed (e.g. DABAL-Et₃ (1, R³,R⁴,R⁵=Et) is a reactive oil while the 1:1 oligomer (5) is a considerably more stable crystalline solid. The only previously described member of this class DABAL-Me: (1 R¹,R²,R³=Me) has far greater stability than that described by Bradley [A. M. Bradford, D. C. Bradley, M. B. Hursthouse, M. Moteiralli, Organometallics 1992, 11, 111-115.] greatly facilitating its handling. A last feature of this aspect of the invention is that, if preferred, crystalline DABAL reagents may be pressed into small pellets (tablets) under pressures greater than 3 atm. to afford tablets of predetermined mass which are useful for controlled delivery of reagent or prolonged storage through blister-packing of the derived tablets. The stability of the pressed tablets can be improved further by use of a polyacrylate coating and/or binding additive (preferably poly-butylmethacrylate). These tablets dissolve on addition to tetrahydrofuran solutions.

No organoaluminium reagent prior to this publication has been used in an asymmetric nickel-catalysed aldehyde addition. No DABAL reagent has been used in any catalytic reaction prior to this disclosure. DABAL reagents will be of high utility in various transition metals catalysed processes including: cross-coupling reactions, conjugate additions, aldol reactions and of utility in their own right in synthetic organic chemistry.

Fujisawa disclosed a racemic process as hereinbefore described. We have found that not only does the use of a chiral catalyst enable the preparation of chiral alcohols, but that the use of a particular class of chiral catalyst enables excellent results. Chiral ligands which may form a chiral catalyst according to the invention are known (I), (II), (III).

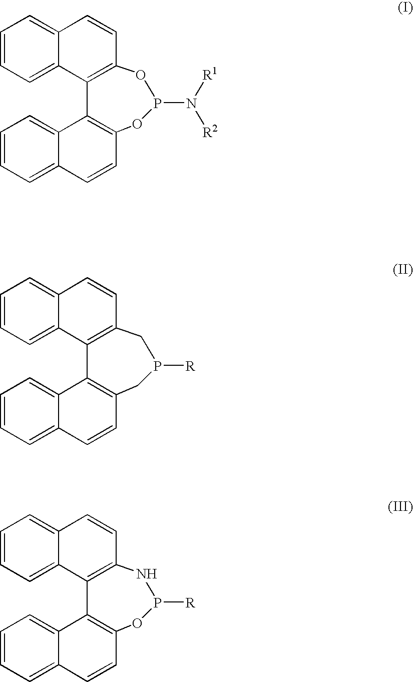

In some embodiments of the present invention use of ligand structures outside those claimed by structures (I), (II), (III) are preferred.

Suitably the chiral ligand is of the formula (26)

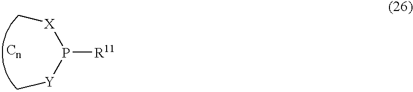

wherein Cₙ together with the X, Y and P atoms forms a ring with 2-4 C atoms which may be substituted or unsubstituted or form a fused or spiro mono or polycyclic aromatic structure, and together with any substitutents or fused or spiro mono or polycyclic aromatic structure comprises 6-45 C atoms and heteroatoms; X and Y are each independently selected from O, S, CH₂, NH, NR²⁰, CR²⁰₂, CR²⁰H where R²⁰ is selected from alkyl, allyl, vinyl, aryl, heteroaryl or substituted variants thereof, wherein substituents are selected from aryl, alkyl, alkoxy, hydroxy, nitrile, halogen or carbonyl and the like, preferably Ph, Ar, OMe, OCOR⁷ etc or R¹¹ is part of a polymeric backbone and X-Cₙ—Y is any aliphatic or aromatic tether that engenders the phosphorus to become chiral at that centre and R¹¹ is selected from H, OH or substituted or unsubstituted C₁₋₂₄ alkyl, alkenyl, aryl, aralkyl, alkaryl, heteroaryl or heterocyclic, or OR⁷, SR⁷, NHR⁷, NR⁷₂ where R⁷ is as defined for R¹¹ or is any linear, cyclic or branched alkyl or aryl capable of bearing additional substituents selected from aryl, alkyl, alkoxy, hydroxy, nitrile, halogen or carbonyl and the like, preferably Ph, Ar, OMe OCOR⁷ etc.

Preferably the tether is as defined in WO 02/04466, the contents of which are incorporated herein by reference. In a particular advantage we have found that the chiral ligand may form a chiral centre through steric factors, for example through substitution of the C moiety of the tether or through the nature of R¹¹ which interacts with the C moiety of the tether.

Preferably the chiral ligand is of the formula (22)

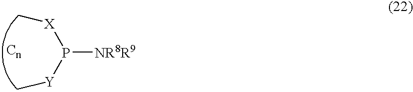

where R⁸ and R⁹ are as defined for R¹¹ or R⁸ and R⁹ form a heterocyclic or heteroaryl ring with the N atom to which they are bound, and are preferably substituted or unsubstituted aryl groups, heteroaryl groups, aliphatic groups or combinations thereof.

More preferably the chiral ligand is of the formula (22a)

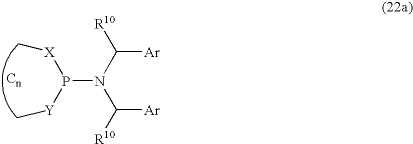

wherein Ar is a substituted or unsubstituted aryl or heteroaryl and Cₙ, X and Y are as hereinbefore defined, and R¹⁰ is as defined for R¹¹ and is preferably alkyl more preferably methyl, or
is of the formula (8)

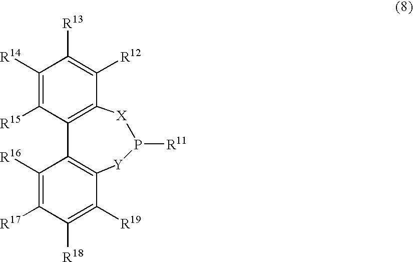

wherein X and Y are each independently selected from O, S, CH₂, NH, NR²⁰, CR²⁰₂, CR²⁰H where R²⁰ is selected from alkyl, allyl, vinyl, aryl, heteroaryl or substituted variants thereof and R¹¹ is as hereinbefore defined, and each R¹²⁻¹⁹ is independently selected from H, R¹¹ as hereinbefore defined, COalkyl, COaryl, OCOAlkyl, OCOAryl, F, Cl, Br, OH, NO₂, Trialkylsilyl, CF₃, CN, CO₂H, CHO, Salkyl, Saryl, SOalkyl, SOaryl, SO₂alkyl, SO₂aryl, SO₃H, SO₃alkyl, SO₃aryl, CO₂NH₂, CONH₂, CONHalkyl, CONH(alkyl)₂, NHCOH, NHCOalkyl, CH═CHCO₂alkyl, CH—CHCO₂H etc and preferably one of ^{R12} to R¹⁵ and R¹⁶ to R¹⁹ is any aryl or heteroaryl group, or wherein any two adjacent groups R¹² to R¹⁹, preferably R¹² and R¹³, R¹³ and R¹⁴, R¹⁴ and R¹⁵ or R¹⁶ and R¹⁷ or R¹⁷ and R¹⁸ or R¹⁸ and R¹⁹ or R¹⁵ and R¹⁶, together with the X—Cₙ—Y structure, form one or more single or fused or spiro aromatic ring structures, and at least one R¹² is not H and is such as to form a chiral centre, or R¹¹ is substituted, preferably is NR⁷₂ or where R⁷ is C₁₋₅alkyl or aryl and is not H, such as to form a chiral centre; or is of formula (8a)

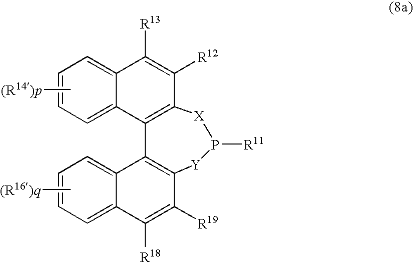

wherein X, Y and R¹¹ and R¹², R¹³, R¹⁸and R¹⁹ are as hereinbefore defined, p and q are each independently zero or a whole number integer from 1 to 4.

Preferably X and Y are respectively O, S; CH₂, O; CHR²⁰, O; C(R²⁰)₂, O; CH₂, S ; or NR²⁰, O. Preferably alkyl and alkenyl comprise 1 to 4 C atoms, whereby an alkenyl group comprises one double bond, and aryl comprises 5 to 7 C atoms, heteroaryl groups comprise preferably one or two N atoms, a N and an O atom or a S and an O atom.

Preferably R¹¹ is optionally substituted as hereinbefore defined and wherein substituents include OR¹¹, SR¹¹, Ph, aryl, alkyl, alkenyl, heteroaryl, NHR⁹, NR⁹R¹⁰ where R⁹ is CR⁶R⁷ and R⁶ and R⁷ are each independently selected from methyl, phenyl, naphthyl, preferably methyl and phenyl respectively.

In a preferred embodiment the chiral ligand is selected from formulae (6), (7), (8), (9), (10) or (11) or (12)

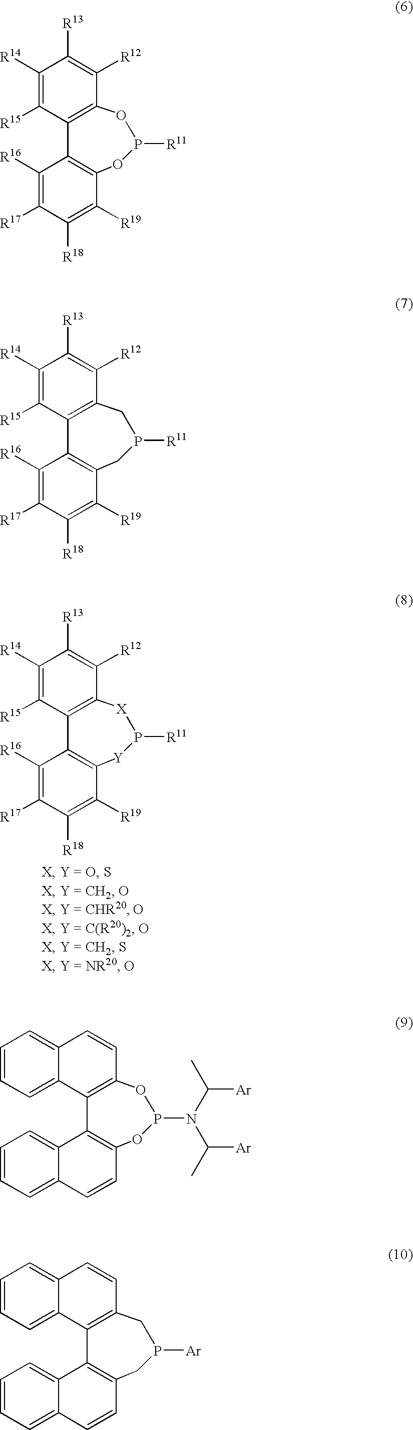

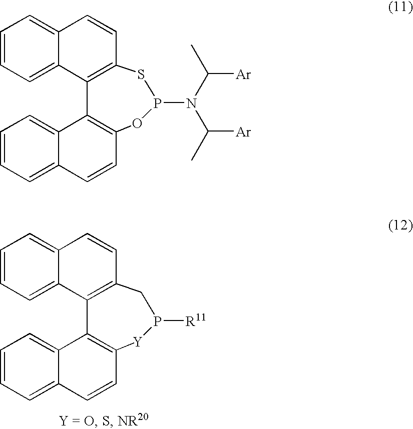

or from formulae (L1-L6) or (23) or (L7-L9)

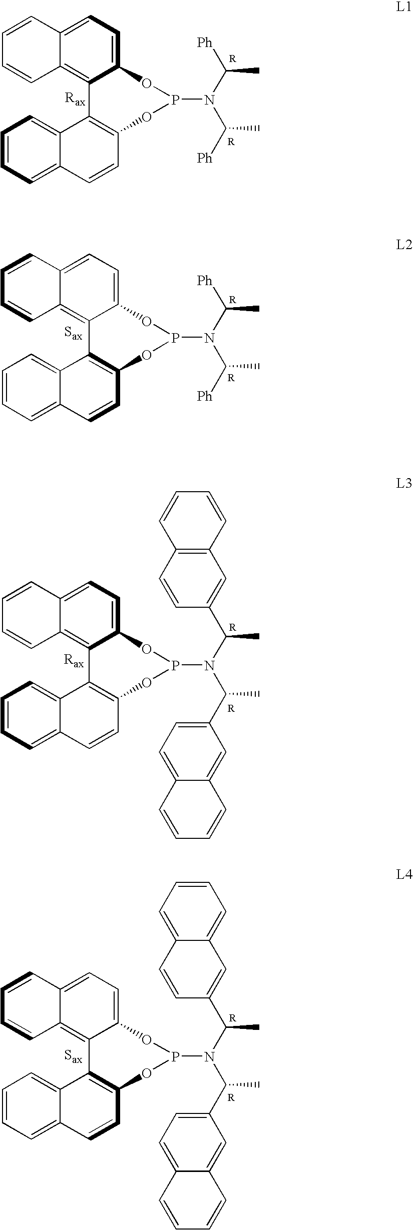

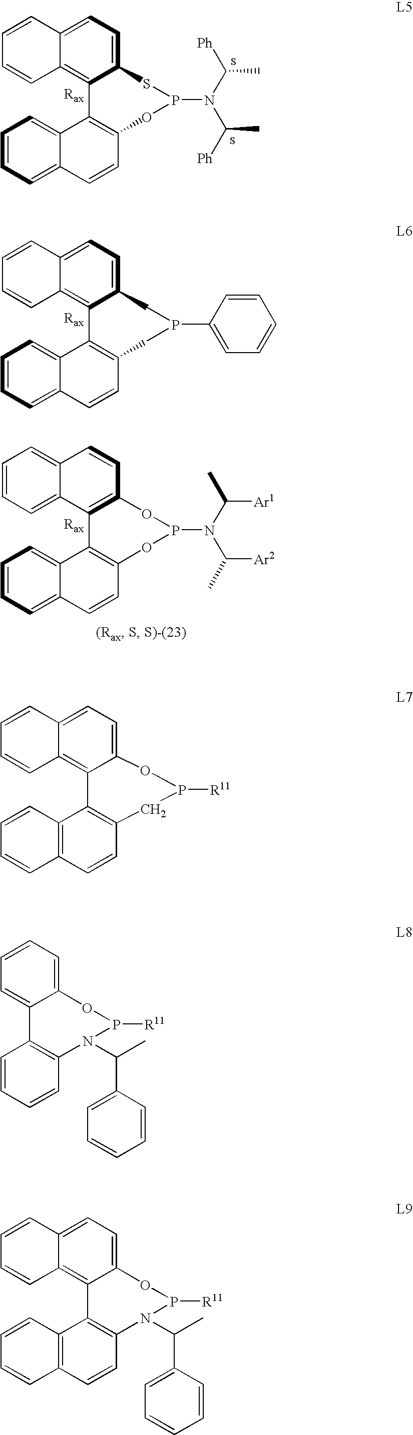

Preferably X and Y are respectively O and S; or CH₂, CHR²⁰ or CR²⁰₂ and O; or NH and O; or NR²⁰ and O; or CH₂, CHR²⁰ or CR²⁰₂ and S; or are both O; or are both CH₂, CHR²⁰ or CR²⁰₂, preferably CH₂, CHR²⁰ or CR²⁰₂ and O; or O and S; or NH and O; or NR²⁰ and O.

The chiral ligand may be in the form of any of its R or S isomers or a combination thereof. The chiral ligand (B) is a chiral phosphoramidite or related phosphane ligand. It will be understood that where one enantiomer or a racemate is represented, either enantiomer is similarly applicable. Structures (6) and (7) are preferred ligand architectures for the catalytic process (where R¹=H, alkyl, alkenyl, aromatic, heteroaromatic, or OR, NHR, NR₂ where R is any linear, cyclic or branched alkyl capable of bearing additional substituents e.g. Ph, Ar, OMe, etc. and R²—R⁹=R¹, OCOAlkyl, OCOAryl, F, Cl, Br, OH, NO₂, Trialkylsilyl, CF₃, CN, CO₂H, CHO, SO₃H, CONH₂ etc.). In an additional preferred embodiment of the invention ligand architectures that allow the phosphorus to become a stereogenic centre are useful, especially structures (8) (where X, Y=O, S; 0,CH₂; S, CH₂). Such C₁ ligands show greater diversity in their symmetry elements than C₂ symmetrical (6)-(7) and are uniquely well disposed to the present reaction.

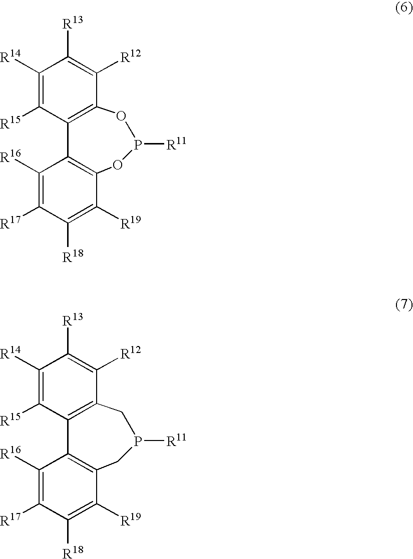

It should be understood that while structures (6) and (7) have been described in prior art and claimed (see DESCRIPTION OF THE PRIOR ART) ligand structures (8) fall outside the remit of the prior art. Within these general architectures (6)-(8) the more specified examples (9)-(12) offer the added advantage of simple synthetic utility and/or commercial availability in the cases of (9)-(10).

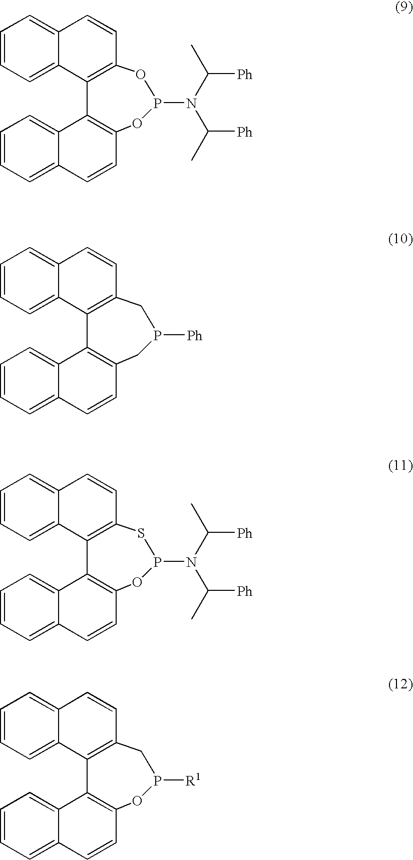

The transition metal source (C). No specialist literature or patents cover the metal compound sources used in this invention which in all cases is well known salts or complexes that are either commercially available or easily prepared by those skilled in the art.

Preferably the Group 5-12 transition metal based catalyst is complexed with a chiral ligand ex situ or in situ.

Preferably the transition metal source is a transition metal compound (MXₓ X=halide or pseudohalide),and required in order to realise viable catalytic activity. Without its presence either no reaction or the formation of side products in low yield results. The chiral ligand (L=6-8) and the metal source react to form a complex (LₙMₙXₙ n=0-4+). Active catalytic systems are produced with many transition metal salts in the presence of the chiral ligands(6)-(8) and either AlR³R⁴R⁵ or DABAL reagents.

Preferably the Group 5-12 transition metal is nickel, preferably Ni(acac)₂.

In the preferred embodiment of the present process for the generation of enantio-enriched secondary alcohols simple nickel(II) salts (Ni^{II}X₂; X=halide or pseudo halide) are of the greatest utility. The highest activity is realised through the use of soluble Ni(acac)₂ and related species. It will be understood that at the onset of catalysis the reduction of the nickel(II) precursor to a catalytically active nickel(0) species takes place followed by complexation of nickel (0) species by the chiral ligand to form an active complex LₙNi (n=1-2). Therefore many nickel(II) or Ni(0) precursors that fulfill this requirement can be identified by those skilled in the art without substantively changing the nature of the catalytic process (e.g. the use of Ni⁰(COD)₂ with the same chiral ligand leads to an essentially identical catalytic system).

In the simplest approach to catalyst generation dry Ni(acac)₂ (0.1 to 1 mol %) is suspended in dry tetrahydrofuran (THF), under an inert atmosphere, and the chiral ligand (0.15 to 2 mol %) added. At either −20° C. (for simple organoaluminium reagents, AlR³R⁴R⁵) or 0 to 5° C. (for DABAL reagents) the organoaluminium reagent is added followed by the substrate (RCHO). The formation of an active catalyst is signalled by the generation of a lemon yellow colour. The reaction is stirred at −20° C. or 5° C. as required by the reagent for 1-3 h (longer times are required for catalyst loadings below 0.5 mol %) and the reaction quenched with aqueous ammonium chloride (sat.) or hydrochloric acid (2 M). Normal extractive workup procedures give the desired secondary alcohol in excellent chemical yield and good to excellent ee value (substrate dependent see DETAILED DESCRIPTION OF INVENTION).

### DETAILED DESCRIPTION OF THE INVENTION

The invention allows the preparation of enantio-enriched chiral secondary alcohols using: (A) the organoaluminium reagent; (B) the chiral ligand; and (C) the transition metal source and associated reaction conditions. Preparations and conditions associated with the invention are described in detail below.

The organoaluminium reagent (A). The present invention uses AlR³R⁴R⁵ as hereinbefore defined or stabilised forms thereof attained by complexation of a suitable additive. AlR³R⁴R⁵, AlR³R⁴X, AlR³X₂ are widely commercially available, known examples of the stabilised forms are exemplified by: (DABCO)(AlMe₃)₂ [A. M. Bradford, D. C. Bradley, M. B. Hursthouse, M. Moteiralli, Organometallics 1992, 11, 111-115]; AlMe₂(CH₂)₃NMe₂ [H. Schumann, B. C. Wassermann, S. Schutte, B. Heymer, S. Nickel, T. D. Seup, S. Wernik, J. Demtschuk, F. Girgsdies, R. Weimann, Z. Anorg. Allg. Chem. 2000, 626, 2081-2095]; AlMe₂X(CH₂)₂NMe₂ (X=O, NR) [D. Gelman, S. Dechert, H. Schumann, J. Blum, Inorg. Chim. Acta 2002, 334, 149-158; H. Schumann, M. Frick, B. Heymer, F. Girgsdies J. Organomet. Chem. 1996, 512, 117-126]. No such reagent has previously been used for an addition to a carbonyl compound by asymmetric catalysis as is the case in the present invention.

The DABAL organoaluminium reagent. The organoaluminium reagent AlR³R⁴R⁵ (R³,R⁴,R⁵=alkyl, alkenyl, alkynyl, allyl, aryl, heteroaryl) may be reacted with an amine NR⁶R⁷R⁸ (R⁶,R⁷,R⁸=alkyl, allyl), directly such that new Lewis acid-Base pairs are formed. Some examples of these reagents can be prepared by those skilled in the art through application of known procedures, for example, Me₃NAlMe₃ [N. Davidson, H. C. Brown, J. Am. Chem. Soc. 1942, 64, 316] for those reagents specifically prepared from DABCO (1,4-diazabicyclo(2.2.2)octane) only one species has been previously reported (DABCO)(AlMe₃)₂ [A. M. Bradford, D. C. Bradley, M. B. Hursthouse, M. Moteiralli, Organometallics 1992, 11, 111-115]. This reagent is referred to as DABAL-Me₃ 1-Me₃ by us. Identical reactivity can be used to generate higher members of the DABAL family 1-R³R⁴R⁵ from appropriate commercial or otherwise prepared organoaluminium—these species have not been reported before. Typical, though not exhaustive, examples of DABAL reagents attained in this way are given in structures (13)-(16). When working with these DABAL reagents in situ further diversity can be introduced by changing the Al:N molar ratio in their preparation either favouring either oligomeric/polymeric reagents (such as 1a or 5 above) or polynuclear species such as 17 (attained through bridging interactions). The propensity to use commercial DABCO for the formation of DABAL reagents (due to DABCO's ready availability) shall not preclude the use of other mono or poly substituted diazabicyclo(2.2.2)octane type amines in the formation of DABAL reagents for special applications such as in structure (1a) above (where R³-R⁶ and n are as hereinbefore defined).

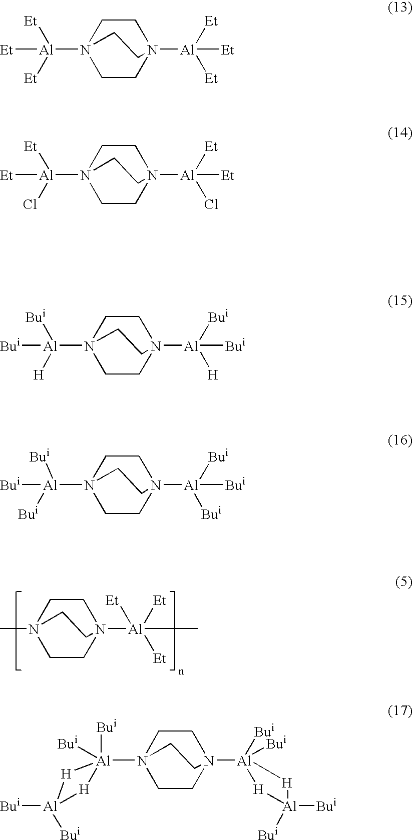

In an alternative modification of the present invention DABAL reagents can be prepared by initially modifying an existing organoaluminium reagent by its reaction with an unsaturated π-hydrocarbon (e.g. a molecule containing one or more C—C double or triple bond before subsequent reaction of the derived organoaluminium reagent with a polyamine, typically DABCO. Many literature methods [overviews: (a) M. Lautens, T. Rovis, Tomislav in Comprehensive Asymmetric Catalysis I-III 1999, Vol. 1 337-348. Springer-Verlag, Berlin, Germany; (b) E. Negishi, D. Y. Kondakov, Chem Soc. Rev. 1996, 25, 417-426] are known to those skilled in the art for the preparation of such species. As shown in the following Scheme, AlHBu¹₂ can be used to hydroaluminate alkenes (e.g. to 18) [e.g. E. Negishi, T. Yoshida, Tetrahedron Letters 1980, 21, 1501-1504], allenes (e.g. to 19) [S. Nagahara, K. Maruoka, Y. Doi, H. Yamamoto, Chem. Lett. 1990, 1595-1598] or alkynes (e.g. to 20) [S. Baba, E. Negishi, J. Am. Chem. Soc. 1976, 98, 6729-6731] prior to addition of DABCO and formation of the DABAL reagent. Similarly, AlMe₃/Cp₂ZrCl₂ may be used to afford carboaluminated alkynes [E. Negishi, N. Okukado, A. 0. King, D. E. van Horn, B. I. Spiegel, J. Am. Chem. Soc. 1978, 100, 2254-2256] prior to DABCO complexation (affording 21).

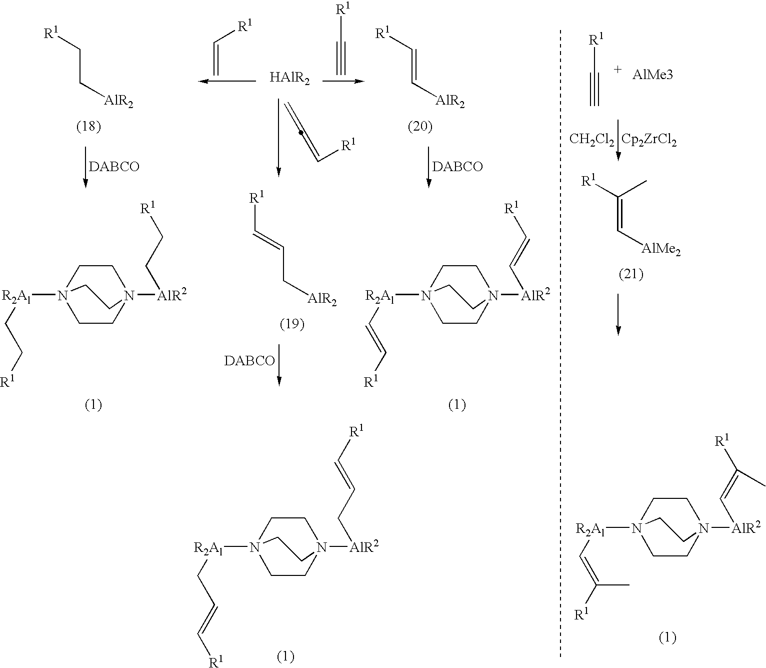

In all these cases however the subsequent complexation of the organoaluminium reagent by polyamines, normally DABCO, has not been previously described. R¹ has been defined hereinbefore.

The presence of the coordinated amine in the DABAL reagent confers a number of modifications to the reactivity of the organoaluminium reagents thus reacted Examples include: reduced pyrophoric mature, increased basisity, crystalline solid formation, retardation of background reactions. All of these features are of utility in aspects of organic synthesis.

While some members of the DABAL reagent family can be handled under normal laboratory conditions some are reactive solids. In one aspect of this invention improved handling and storage characteristics can be attained through compressing the materials into pellets of reduced surface area. This process may be carried out in manual apparatus or on appropriately automated machinery designed for the pressing of tablets. Adhesion of the DABAL particles can be improved by use of an appropriate polymer such as polystyrene or a polyacrylate or polymethacrylate. In a preferred embodiment of this aspect of the invention poly (butylmethacrylate) is used. The melting point and T_{g} temperature of this compound also making of use for sealing the press-derived pellets against atmospheric oxygen and moisture by dipping in polymer melts or solutions.

In preferred embodiments of the present invention the parent organoaluminium reagent and DABCO are reacted together in a solvent which they are both soluble at a temperature in the range −10 to +5° C., but normally 0° C., at the preferred stoichiometry (in the range 4:1 to 1:1 organoaluminium reagent: DABCO but normally 2:1) and the solution used directly. Alternatively the derived DABAL reagent precipitates form the reaction mixture or can be induced to crystallise by cooling or by addition of hexanes or pentanes. Preferred solvents for the preparation of DABAL reagents are: toluene, ethers (including diethyl ether and tetrahydrofuran) or ether/hydrocarbon mixtures. In the cases of isolated DABAL reagents it is preferable to wash the derived solid in an inert solvent (normally toluene, hexanes or pentanes) to ensure all traces of uncoordinated organoaluminium reagents are removed.

It will be understood that these preferred embodiments do not exclude the formation and use of other organometallic adducts of poly amines, especially DABCO, such as (AlR³R⁴X)₂(DABCO), (AlR³X₂)₂(DABCO), (R³,R⁴=alkyl, alkenyl, alkynyl, aryl, allyl, heteroaryl; X=halide pseudohalide) and their use in subsequent organic synthesis and associated catalytic reactions.

The chiral Vigand (B). The chiral ligand in the catalyst can be a phosphoamidite (6, R¹¹=NR₂) or related phosphane ligand of type (7) or (8) above.

The chiral ligand (B). The invention reported herein can use various chiral ligands and some of these are already known. These known structures are (I), (II) and (III) above. Structure (I) is claimed by Feringa through the DSM company [M. van den Berg, A. J. Minnaard, B. Feringa, J. Gerardus de Vries, PCT Int Appl. 2002, 27 pp. CODEN: PIXXD2 WO 2002004466 A2 20020117 (DSM N. V., Netherlands)]. Structure (II) is part of the subset of ligand structures claimed by Degussa [M. Beller, K. Junge, A. Monsees, T. Riermeier, H. Trauthwein, PCT Int Appl. 2003, 27 pp. CODEN: PIXXD2 WO 2003033510 A1 20030424 (Degussa A. -G., Germany)] or Zhang (Structure II, R=NR₂) [X. Zhang, U.S. Pat. Appl. Publ. 2004,15 pp. CODEN: USXXCO US 2004072680 A1 20040415 (Penn State Res. Found., USA)]. Structure (III) also claimed by Zhang [X. Zhang, PCT Int. Appl. 2001, 93 pp. CODEN: PIXXD2 WO 2001000581 A1 20010104 (Penn State Res. Found., USA)] while not used in the present work it is of relevance in the discussion of preferred ligand types in one preferred embodiment of the present catalyst system.

If a phosphoramidite ligand (6, R¹¹=NR₂) is used in the catalyst composition of the invention may be any phosphoramidite, such as those disclosed in International Patent Application Publication WO 02/04466, which is hereby incorporated by reference in its entirety. Preferably, the phosphoramidite ligand has the general structure (22).

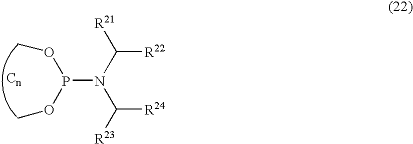

In structure (22), O—Cₙ—O is an aliphatic or aromatic diolate. R²¹, R²², R²³, and R²⁴ are preferably substituted or unsubstituted aryl groups, substituted or unsubstituted heteroaryl groups, substituted or unsubstituted aliphatic groups, or combinations of such groups. However, at least one of R²¹, R²², R²³, or R²⁴ must be a substituted or unsubstituted aryl or heteroaryl group.

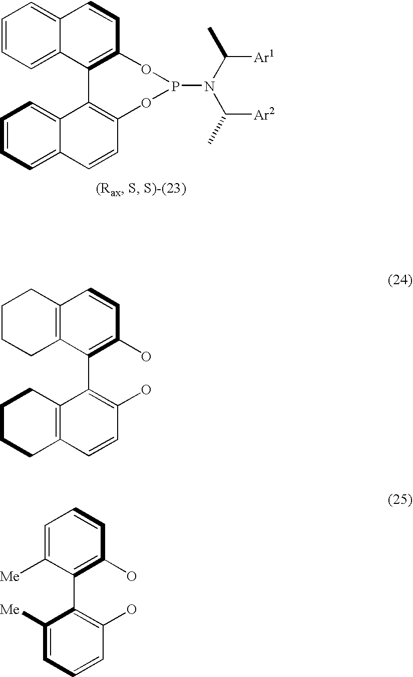

In one embodiment, a preferred O—Cₙ—O group is an aromatic group having the general structure (23). In this general structure, Ar¹ and Ar² are individually aryl, substituted aryl, or heteroaryl. Examples of useful O—Cₙ—O groups having this general structure include, but are not limited to (24)-(25). It will be understood by those skilled in the art that these structures may be in any combination of R or S enantiomers, and that both enantiomers may be implemented in the present invention.

If a phosphane ligand (7) is used in the catalyst composition of the invention may be any phosphane of this type, such as those disclosed in International Patent Application Publication WO 2003033510 or US Patent Application Publication US 2004072680 both of which are hereby incorporated by reference in their entireties. Preferably, the phosphane ligand has the general structure (7) where R¹¹ is an aryl, substituted aryl, or heteroaryl unit.

If a chiral at P-ligand (8) is used preferably, the ligand has the general structure (26). In this structure, X—Crₙ—O is an aliphatic or aromatic linker where X=S, CH₂, CHR²⁰, CR²⁰₂, NR²⁰ and Y=O, S. CH₂ such that a chiral centre is generated at phosphorus.

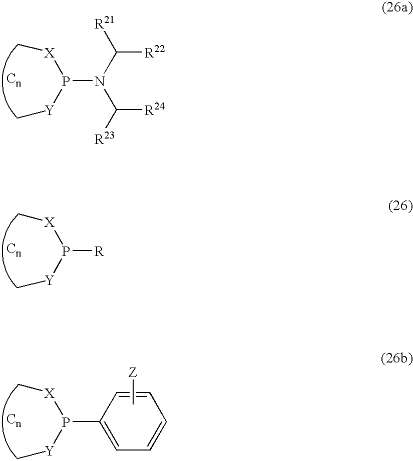

The group R in Structure (26) is preferably an amide derived unit (Structure 26a) where R²¹, R²², R²³, and R²⁴ are preferably substituted or unsubstituted aryl groups, substituted or unsubstituted heteroaryl groups, substituted or unsubstituted aliphatic groups, or combinations of such groups. However, at least one of R²¹, R²², R²³, or R²⁴ must be a substituted or unsubstituted aryl or heteroaryl group. Alternatively, it is preferable to have R¹¹ in Structure (26b) as an aryl, substituted aryl, or heteroaryl unit where Z represents mono or poly modification of the aryl or heteroaryl (Structure 26b). These preferred embodiments of the invention do not indicate that there is a restriction on the use of other types of R¹¹ substituent in structure (26) i.e. where R=H, alkyl, alkenyl, aromatic, heteroaromatic, or OR, NHR, NR₂ where R is any linear, cyclic or branched alkyl capable of bearing additional substituents e.g. Ph, Ar, OMe, etc. ). It will be understood by those skilled in the art that these structures may be in any combination of R or S enantiomers, and that both enantiomers may be implemented in the present invention.

In one embodiment, a preferred X—Cₙ—Y group is an aromatic linker having the general structure (27), (28) or (29).

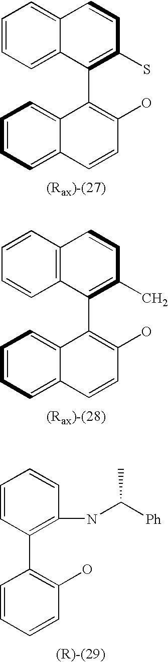

Examples of useful X—Cₙ—Y groups having this general structure include, but are not limited to (8).

The phosphoramidites (e.g. 22) and the thiophosphoramidite (e.g. 26a X=S, Y=O) of the present invention may be produced using know procedures, such as those described by Alexalds (A. Alexalds, and co-workers, Synlett, 2001, 1375. herein incorporated by reference in its entirety (see also EXAMPLES). The fragment (27) is available through known procedures [S. Azad et al., J. Chem. Soc., Perkin Trans 1, 1997, 687-694]. Preparation of ligand (26a-b) requires metallation of the parent methyl compound (2-hydroxy-2′methyl-1,1′-binaphthyl [compound 2 (X=OH) in: J. Blum, D. Gelman, Z. Aizenshtat, S. Wernik, H. Schumann, Tetrahedron Lett 1998, 39, 5611-5614] under vigorous basic conditions [e.g. L. Brandsma, Preparative Polar Organometallic Chemistry, Vol 2, Springer-Verlag, Berlin ISBN 3-540-52749-4] and subsequent trapping with (LG)₂PR¹¹ or (LG)₂PN(CHR²¹R²²)(CHR²³R²⁴) reagents (LG=a suitable leaving group, such as Cl, Br, I, OEt, OMe, OPh, OAr, OR) (e.g. to give8X=CH₂,Y=O).

In a further aspect of the invention there is provided a process for the preparation of a chiral ligand as hereinbefore defined comprising reaction of a compound of formula (30)

| | |
|---|---|
| | (30) |
| 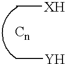 | |

| X | Y |
|---|---|
| O | S |
| O | CH₂ |
| O | CHR²⁰ |
| O | CR₂²⁰ |
| O | NR²⁰ |

and (LG)₂PR¹¹ or (LG)₂PN(CHR²¹R²²)(CHR²³R²⁴) reagents (LG=a suitable leaving group, such as Cl, Br, I, OEt, OMe, OPh, OAr, OR) and in the presence of a base or nBuLi/TMEDA.

An intermediate of formula (30) wherein X=O and Y=S

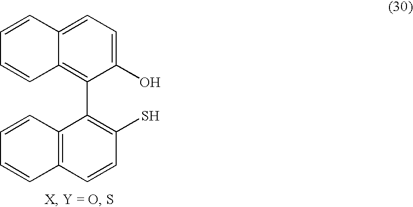

may be prepared as disclosed in S. M. Azad, S. M. W. Bennett, S. M. Brown, J. Green, E. Sinn, C. M. Topping, S. Woodward, J. Chem. Soc., Perkin 1 Trans. 1997, 687-694.

An intermediate of formula (30) wherein Y is CH₂, CHR²⁰ or CR²⁰₂ may be formed by the reaction of a disulfonyl precursor (31)

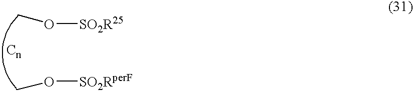

where R^{perF} is any perfluorinated C₁₋₅ alkyl, preferably CF₃ and R²⁵ is alkyl, alkenyl, aryl for example phenyl, 4-tolyl, tBu or CH₃
to form the intermediate wherein X and Y are respectively O and CH₂, CHR²⁰, CR²⁰₂, with a source of organometallic anions, such as R³R⁴CHMgBr preferably R³ and or R⁴=H, Me, in the presence of a nickel catalyst with subsequent hydrolysis to OH, e.g. with KOH;
An intermediate of formula (30) wherein X and Y respectively are O and NR²⁰ may be formed by the reaction of the disulfonyl precursor (31) as defined, with H₂NR²⁰ and Pd₂(dba)₃, Xantphos (9,9-Dimethyl-4,5-bis(diphenylphosphino)xanthene (Chemical Abstracts No. [161265-03-8]) and LiHDMS.

Intermediates of formula (31) may be formed by the reaction of a diol precursor (32)

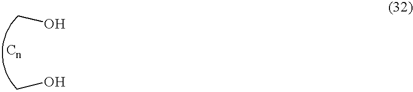

with R²⁵SO₂Cl followed by R^{perF}SO₂(LG) where LG is any viable leaving group (e.g. F, Cl, OSO₂R^{perF}, etc.).

Exemplary intermediates include structures (30), (31) and (32) of formulae (33a-b), (34a-b), (35), (36a-c), (37) prepared by these techniques, and intermediate (LG)₂PN(CHR²¹R²²)(CHR²³R²⁴) prepared as described hereinbelow.

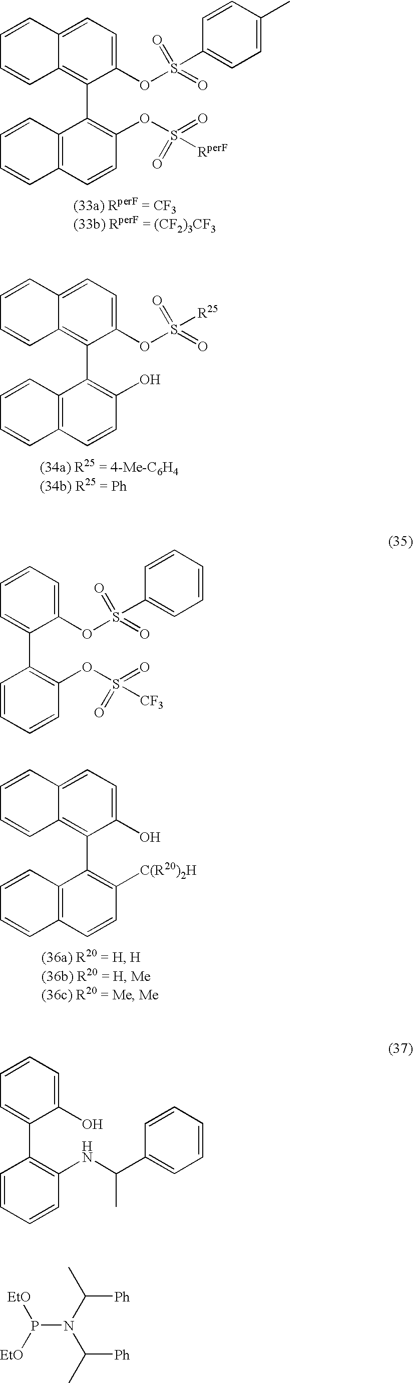

In a further aspect of the invention there is provided a novel intermediate of formula 30, specifically of formula (33a-b), (34a-b), (35), (36b-c) or (37).

The transition metal source and associated reaction conditions (C). In a final aspect of the present invention, the presence of a transition metal compound is required in order to realise viable catalytic activity. Provided AlR¹R²R³, or their derived DABAL reagents (in whatever form) are present active catalytic systems are produced with many transition metal salts and complexes including: [Cu(MeCN)₄]BF₄, Cu(TC), [RhCl(COD)]₂, [Rh(OMe)(COD)]₂, [IrCl(COD)]₂, Pd(OAc)₂, Pd₂(dba)₃ (where TC=the anion of 2-thiophenecarboxylic acid, COD=1,5-cyclooctadiene and dba=dibenzylideneacetone). It will be understood that such complexes formed from these precursors and the chiral ligands (6-8) have high potential for use in asymmetric catalysis.

In the preferred embodiment of the present process for the generation of chiral secondary alcohols simple nickel(II) salts (Ni^{II}X₂; where X is Cl, Br, I, BF₄, PF₆, OSO₂CF₃, OSO₂Ar, OAc, etc.) generate the most active system for the addition of AlR¹R²R³ or derived DABAL reagents to aldehydes. The highest activity is realised through the use of soluble Ni(II) complexes of which the preferred source, among many, is Ni(acac)₂ due to its ready commercial availability. It will be understood that prior to catalysis reduction of the nickel(II) precursor to a catalytically active nickel(0)L* (L*=chiral ligand) species takes place. Therefore many nickel(II) precursors that fulfil this requirement can be identified by those skilled in the art without substantively changing the nature of the catalytic process (e.g. the use of Ni⁰(COD)₂ with the same chiral ligand would be expected to lead to an essentially identical catalytic system).

In a preferred procedure for the formation of active catalysts the nickel precursor (typically anhydrous Ni(acac)₂) is dissolved in a solvent in the presence of the chiral ligand (6)-(8). The solvent may be toluene, dichloromethane, benzene, xylene, dichloroethane, or any ethereal solvent (R¹OR²; R¹, R²=alkyl, aryl, etc.). In a preferred approach the catalytic procedure dry, O₂ free, tetrahydrofuran is used such that the concentration of the final added aldehyde substrate will be 0.1 and 2.0 M and the molar ratio of [RCHO]/[Ni] will be between 1000 and 100 at ambient temperature (0.1 to 1 mol % Ni). The concentration of the chiral ligand should be kept at least equal to that of the nickel and preferably in the range [chiral ligand]:[Ni]=1.0 to 2.0 (0.1 to 2 mol % ligand). The reaction is equilibrated at the desired temperature in the range −40 to +22° C. and the dry substrate aldehyde added in one portion. While the reaction is tolerant of small amounts of moisture and atmospheric oxygen gross contamination of the reaction mixture with either of these contaminants is not conducive to attaining optimal results.

Suitably in the process of the invention the carbonyl group is present in a prochiral aldehyde and the product of the reaction is a chiral secondary alcohol or a mixture of the two enantiomeric forms of a chiral secondary alcohol in any proportion.

Preferably a substrate that contains a carbonyl group is R¹C(═O)R² where R¹ and R² are each independently selected from linear, cyclic or branched alkyl, alkenyl, alkynyl, allyl, aromatic or heteroaromatic and can contain one or more functional groups or substituents selected from CO₂R, COR, CONH₂ SO₃R, OH, OR, NH₂, NHR, Cl, Br, I, NO₂, alkenyl, allyl, alkynyl, aryl, heteroaryl, and the like, wherein substituents as side chains may bear functional groups as defined, or R² is H.

Preferably in the process as hereinbefore defined the solvent is ethereal such as tetrahydrofuran.

Any organoaluminium reagent as hereinbefore defined may be used in the reaction of aldehydes. Preferably a DABAL reagent of formula (1) or (1a-b) as hereinbefore defined is used in the reaction of aldehydes.

In one preferred method of operation (called Method “A” in Tables 1-2) the reaction mixture, as generated above, is cooled to 5° C. and any DABAL reagent (1-4, but normally 1, R³R⁴R⁵=Me₃) added as a solid of THF solution added to the reaction mixture. After addition of the DABAL reagent the reaction is stirred at 5° C. for 30 min to 3 h until completion of the reaction of the reaction is indicated by chiral GC, NMR or TLC. The reaction quenched with saturated aqueous ammonium chloride solution and the product extracted by the normal methods of organic chemistry. Alternatively the amount of conversion and chemical yield could be determined by quantified chiral GC using an internal standard. The overall transformation is that of a prochiral aldehyde into the chiral sec-alcohol R¹R²CHOH. These transformations are exemplified for the preparation of Typical yields and ee values are shown in Table 1-2.

In a second preferred method of operation (called Method “B” in Tables 1-2) the mixture of nickel(II) source (typically Ni(acac)₂) and the chiral ligand dissolved in a suitable solvent, the mixture being generated as above, is cooled to −20° C. and any organoaluminium reagent AlR³R⁴R⁵ (but often AlMe₃) added slowly as either the neat reagent or a suitable solution added to the reaction mixture. After addition of the Al R³R⁴R⁵ reagent the reaction is stirred at −20° C. for 1 min to 8 h until or until completion of the reaction of the reaction is indicated by chiral GC, NMR or TLC. The reaction quenched with saturated aqueous ammonium chloride solution and the product extracted by the normal methods of organic chemistry. Alternatively the amount of conversion and chemical yield could be determined by quantified chiral GC using an internal standard. The overall transformation is that of a prochiral aldehyde into the chiral sec-alcohol R¹R²CHOH. These transformations are exemplified for the preparation of Typical yields and ee values are shown in Table 1-2.

The substrates for the reaction are carbonyl compounds SC: R¹C(═O)R² where R¹ and R² are defined thus: R¹=alkyl, alkenyl, alkynyl, allyl, aromatic, heteroaromatic, group that is linear, cyclic or branched. The main substituent (R¹ in R¹C(═O)R²) can contain one or more functional groups (CO₂R, COR, CONH₂ SO₃R, OH, OR, NH₂, NHR, Cl, Br, I, NO₂, alkenyl, allyl, alkynyl, aryl, heteroaryl, etc.), side chains, or side chains bearing functional groups. R²=H or R¹. In the preferred embodiment of the invention the nickel-catalysed additions or organoaluminium reagents described here are employed with aldehydes R¹C(═O)H leading directly to non racemic chiral secondary alcohols PA: R¹R²R³COH (e.g. PA: R²=H, R³=Me; or PA: R²=H, R³=Et) on workup of the reaction mixture.

In a further aspect of the invention there is provided a novel organometallic compound complexed with a polyamine as hereinbefore defined and as herein disclosed in the description, claims, examples and/or figures.

In a further aspect of the invention there is provided a chiral ligand of formula (26)

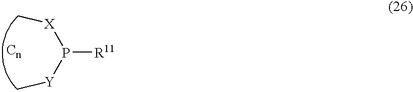

as hereinbefore defined with the proviso that when X is O and Y is O, R¹¹ is not NR⁸R⁹ wherein R⁸ and R⁹ are H, optionally substituted alkyl, aryl, aralkyl, alkaryl or form a hetero ring with the N atom (see structure I, above), or when the ligand is of formula (8)

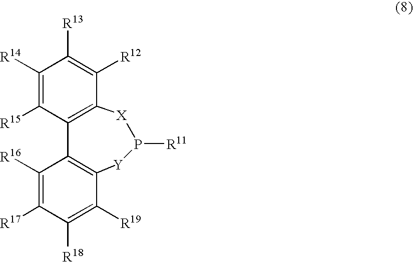

as hereinbefore defined, and one of X and Y is CH₂, the other of X and Y is not CH₂ (see structure II above), or when the ligand is of formula (26) or (8) or when Cₙ is unsubstituted naphthyl, Y is O, X is NH, R¹¹ is not alkyl, aryl, arylene, substituted aryl, heteroaryl, phenol, ferrocene, or aryl carboxylate and in particular phenyl (see structure III above).

In a further aspect of the invention there is provided a catalytic composition comprising an organoaluminium compound as hereinbefore defined.

In a further aspect of the invention there is provided a catalytic composition comprising a chiral ligand as hereinbefore defined.

In a further aspect of the invention there is provided a catalyst composition as hereinbefore defined in which the chiral ligand is complexed with a transition metal containing compound.

Preferably catalyst composition comprises the chiral ligand of formula (26) as hereinbefore defined complexed with a transition metal containing compound, preferably is nickel(I), with the proviso that when X and Y are both O and R¹¹ is NR⁸R⁹, the metal is not rhodium or ruthenium, or with the proviso that when the ligand is of formula (8)

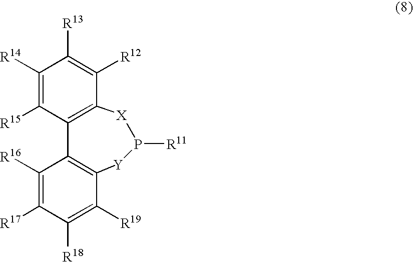

as hereinbefore defined, and one of X and Y is CH₂, the other of X and Y is not CH₂ (see structure II above), or when the ligand is of formula (26) or (8) or when Cₙ is unsubstituted naphthyl, Y is O, X is NH, R¹¹ is not alkyl, aryl, arylene, substituted aryl, heteroaryl, phenol, ferrocene, or aryl carboxylate and in particular phenyl (see structure III above)).

Preferably in the catalytic composition the chiral ligand is complexed with a nickel containing compound.

In a further aspect of the invention there is provided the use of the organometallic compound as hereinbefore defined in cross-coupling, conjugate additions, aldol and related transition metal-ligand promoted processes. Preferably in the presence of Pd and organoaluminium—polyamine complex as hereinbefore defined, methylated products are attained by cross coupling reaction of haloaryl starting material. Between 0.5-0.8 equiv organoaluminium—polyamine complex, eg DABAL-Me₃, ensured complete conversion of aryl bromides and iodides. Selective methylation of aryl-iodide bonds in the presence of acylchlorides was possible.

TABLE
Cross coupling of aryl halides with DABAL-Me₃

| | | Amount |
|---|---|---|
| Starting material | | DABALMe₃, |
| Yield/% | Product | equiv |
| 1-Bromonaphthylene | 1-methylnaphthylene | 0.8 |
| 71 | | |
| 2-Bromonaphthylene | 1-methylnaphthylene | 0.8 |
| 84 | | |
| 2-Bromonaphthylene | 1-methylnaphthylene | 0.5 |
| 97 | | |
| 1-chloro-3-iodobenzene | 1-chloro-3-methylbenzene | 0.8 |
| 96 | | |
| 1-bromo-2-nitrobenzene | 2-nitrotoluene | 0.8 |
| 50 | | |

Conditions: Ratio of DABAL-Me₃/ArX/Pd(PPh₃)₄ 0.5-0.8 mmol/l/1.0 mmol/l/3 mol %, 4 h reflux in THF (3 mL).

In a further aspect of the invention there is provided the use of the process, organometallic compound, chiral ligand or composition as hereinbefore defined in 1,2-addition of an organometallic carbon nucleophile to a carbonyl containing compound.

In a further aspect of the invention there is provided a chiral alcohol moiety obtained with the process or entity as hereinbefore defined or a novel chiral alcohol as herein described in the description, examples or figures.

### EXAMPLES

Preparation of DABAL-Me₃ 1-Me₃. Neat AlMe₃ (4.5 g, 62.5 mmol) was added to a solution of freshly sublimed DABCO (3.4 g, 34.7 mmol) in toluene (30 ml) at 0° C. leading to copious precipitation of a white solid. The precipitate was allowed to settle and the supernatant toluene removed by cannula. Dry diethyl ether (20 ml) was added and swirled with the solid. Solid 1-Me₃ was again allowed to settle and the supernatant liquid cannulared off. The ether wash was repeated four times before the residual slurry was evaporated to dryness under vacuum to yield 1-Me₃ directly 4.5-6.1 g (60-81%); its spectroscopic properties were as described.^{[5]} DABAL-Me₃ 1 stored in screw-top vials under an argon blanket was still active after at least 4 months. [Warning: while we have encountered no problems with this reagent on up to 25 g scales we recommend caution on its initial handling. For example, if the ether washing procedure outlined above is not carried out properly traces of remaining free AlMe₃ can lead to very reactive samples of 1-Me₃. Deliberate addition of water to 1-Me₃ causes strong exotherms and methane liberation; 1-Me₃ ignites on tissue paper especially on “damp” days.]. DABAL-Et₃ 1-Et₃ was prepared by mixing 2:1 molar quantities of AlEt₃ and DABCO in THF it was used in situ. Evaporation of the THF solvent under reduced pressure afforded a reactive oil. Reaction of a 1:1 molar ratio of AlEt₃ (4 mmol) and DABCO (4 mmol) in pentane (30 mL) led, on standing of the mixture, to the formation of large colourless plates of 5 in 50-70% yield. Compound 5 could be handled only very briefly in air. DABAL reagents from other commercial organoaluminium reagents were prepared in situ by similar methods.

Preparation of DABAL-Buⁱ₂H 2. Neat AlHBuⁱ₂ (0.50 g, 3.52 mmol) was added to a solution of freshly sublimed DABCO (0.17 g, 1.76 mmol) in THF (3 ml) at 0° C. an immediate reaction. The reagent was used as attained.

Preparation of DABAL-Buⁱ₂(crotyl) 3. A solution of MeCH=CHCH₂AlBuⁱ₂ (1.0 mmol) in hexane (5 mL) was prepared from methylallene and DIBAL using a standard approach [M. Monyuny, J. Gone, Tetrahedron Left. 1980, 21 51-54.]. Freshly sublimed DABCO (56 mg, 0.5 mmol) was added to the mixture to form a colourless solution. The reagent was used as attained.

Preparation of DABAL-Me₂(vinyl) 4. A solution of PhMeC=CHAlMe₂ (1.0 mmol) in dichloromethane (5 mL) was prepared from phenylacetylene AlMe₃ and Cp₂ZrCl₂ [E. Negishi, N. Okukado, A. O. King, D. E. van Horn, B. I. Spiegel, J. Am. Chem. Soc. 1978, 100, 2254-2256]. Freshly sublimed DABCO (56 mg, 0.5 mmol) was added to the mixture to form a colourless solution. The reagent was used as attained. Equivalent structures were prepared by hydroalumination of terminal alkynes with DIBAL.

Preparation of pelletised DABAL-Me₃ 1. The base of a mechanical die featuring 9 ports was brim filled with DABAL-Me₃ 1, the top plate secured and the die placed in a hydraulic press. The die was subjected to 3-9 atm. pressure for 1-2 min, the pressure released and the die disassembled. The resulting 9 pellets of DABAL-Me₃ 1 (80±1.5 mg; 0.31 mmol, 0.63 mmol in AlMe₃). The structural integrity of the pellets and shelf-life was improved by brief dipping of the pellets into molten polybutylmethacrylate (at 40° C.) followed by immediate cooling at room temperature.

Representative Preparation of P-Chiral Type Ligand Class 8 wherein X=S, Y=O, R¹¹=N(CHMe(Ph)₂)₂.

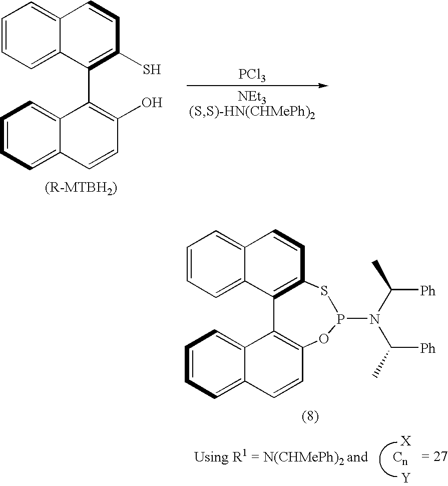

A solution of (S,S)—NH(CHMePh)₂ (0.19 g, 0.85 mmol) and triethylamine (134 μL, 0.97 mmol) in toluene (1 mL) was added dropwise to a solution of PCI₃ (744 μL, 0.85 mmol) under an inert atmosphere. The mixture was stirred at 70° C. (6 h) and then allowed to cool to room temperature after which additional triethylamine (270 μL, 1.43 mmol) was added dropwise. The reaction mixture was cooled to −78° C. and a solution of (Rₐ)-monothiobinaphthol (MTBH₂) (0.85 mmol) in a mixture of toluene (2.5 mL) and THF (0.3 mL). The mixture was allowed to warm to room temperature slowly and stirred for an additional 12 h. The reaction was quenched with ice water, extracted in the normal way and the ligand isolated by column chromatography (0.34 g, 73%). [α]_{D}−264 (c=0.61, CHCl₃), ¹H NMR (270.2 MHz, CDCl₃): δ=8.31 (d, J=9.7 Hz, 1 H, Ar), 8.25-8.14 (m, 4 H, Ar), 7.89 (d, J=9.7 Hz, 1 H, Ar), 7.74-7.60 (m, 2 H, Ar), 7.50-7.23 (m, 14 H, Ar), 7.64 (br, 1 H, CHPh), 1.55 (d, J=7.8 Hz, 6 H, CHMe). ¹³C NMR (67.9 MHz, CDCl₃): δ=149.5, 149.3, 143.3, 137.9, 132.8, 132.7, 132.4, 130.0, 129.7, 129,3, 128.3, 128.2-128.0 (m, 7 C), 127.8, 127.0, 124.5, 124.4, 122.1, 56.0 (CHMe), 55.8 (CHMe), br 22.5 (CHMe). ³¹P NMR (162.0 MHz, CDCl₃): δ=154.8. [HRMS (+ES): found: 556.1852. Calcd. for C₃₆H₃₀NOPS 556.1864].

Representative Preparation of P-Chiral Type 8 Ligand wherein X and Y are O and CH₂.

The compound was prepared via the intermediary of (33a) and (36a), by the chemistry of the following Scheme

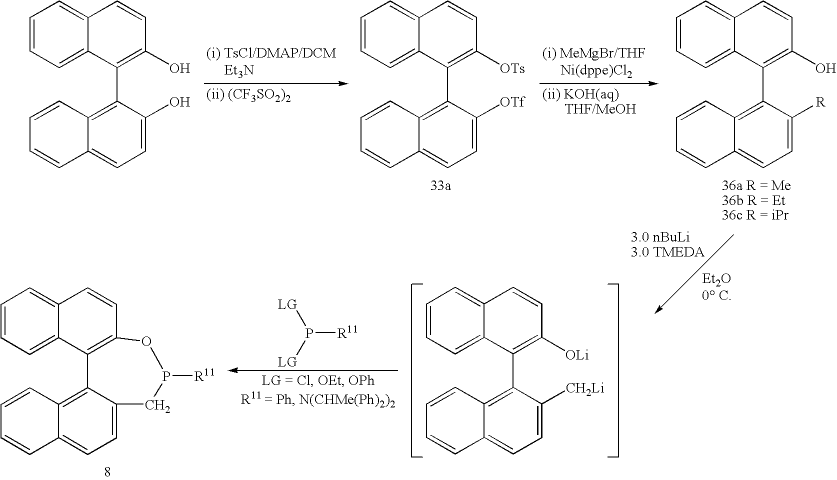

Preparation of (33a): Enantiomerically pure (S)-Binol (5.11 g, 17.85 mmol), p-toluenesulfonyl chloride (3.40 g, 17.85 mmol) and DMAP (0.35 g, 2.86 mmol) were dissolved in a round-bottomed flask with 50 mL of freshly distilled CH₂Cl₂ and the resulting solution cooled to 0° C. Neat Et₃N (8.4 mL, 53.5 mmol) was added drop wise to the solution. The reaction mixture was stirred for 12 hours at room temperature. After this time, trifluoromethanesulfonic anhydride (5.54 g, 19.64 mmol) was added drop-wise at 0° C. to the reaction mixture and stirring continued for another 12 hours. The organic layer was washed with HCl 1 M (3×100 mL) and with water (3×100 mL), dried over MgSO₄ and the solvent removed under vacuum. Recrystallization from boiling MeOH or from CH₂Cl₂/Hexane (ca. 1:6) afforded (33a) as colourless microneedles (9.89 g, 17.28 mmol, 97%). M.p. ° C. [α]_{D}²⁵+108 (c=1.00, CHCl₃). ¹H NMR (400.1 MHz, CDCl₃): δ=8.07 (d, J=8.9 Hz, 1 H, 4-H), 7.97 (d, J=8.1 Hz, 1 H, 4′-H), 7.95 (d, J=8.9 Hz, 1 H, 5-H), 7.89 (d, J=8.2 Hz, 1 H, 5′-H), 7.78 (d, 1 H, J=9.1 Hz, 3-H), 7.53-7.46 (m, 2 H, 6 or 6′ or 7 or 7′-H) overlapped by 7.48 (d, J=9.1 Hz, 1 H, 3′-H), 7.28 (ddd, J₁=1.3 Hz, J₂=6.9 Hz, J₃=8.4 Hz, 6 or 6′ or 7 or 7′-H), 7.22 (ddd, J₁=1.2 Hz, J₂=6.9 Hz, J₃=8.4 Hz 6 or 6′ or 7 or 7′-H) 7.08 (app. d, J=8.4 Hz, 2 H, Ts-o), 7.05 (d, J=8.5 Hz, 1 H, 8 or 8′-H), 6.98 (d, J=8.5 Hz, 1 H, 8 or 8′-H), 6.74 (app. d, J=7.9 Hz, 2 H, Ts-m), 2.22 (s, 3 H, Me). ¹³C NMR (100.6 MHz, CDCl₃): δ=145.9, 145.2, 144.4, 133.1, 132.9, 132.7, 132.0, 131.8, 131.2, 130.8, 129.1 (2 C, Ts-o), 129.0, 128.2, 127.9, 127.4, 127.2 (2 C, Ts-m), 127.1, 126.6, 126.5, 126.3, 124.6, 122.1, 121.4, 119.3, 118.0 (q, J_{CF}=320 Hz, CF₃), 21.6 (CH₃ Ts). ¹⁹F NMR (282.4 MHz, CDCl₃): δ=−75.7 (s, CF₃). MS(FAB): m/z (%) 572 [M]⁺ (100), 440 [M-133 (CF₃O₂S)+1]⁺ (4), 418 [M-155 (C₇H₇O₂S)+1]⁺ (44). C₂₈H₁₉F₃O₆S₂ (572.06): calcd. C 58.7, H 3.3; found C 58.5, H 3.3% [HRMS (FAB): found: 572.0579. Calcd. for C₂₈H₁₉F₃O₆S₂ 572.0575]. The constitution of the compound was also proved by X-ray crystallography.

Similarly prepared were (33b) above and (35).

The initial reaction of the HO—Cₙ—OH tethers is chemospecific for the preparation of the monosulfur esters as shown by the isolation of (34a), [structure above, confirmed by X-ray crystallography].

Selected data for (33b): ¹H NMR (400.1 MHz, CDCl₃): δ=8.06 (d, J=9.0 Hz, 1 H, 4-H), 7.98 (d, J=9.2 Hz, 1 H, 4′-H), 7.94 (d, J=-8.4 Hz, 1 H, overlapped 5-H), 7.88 (d, J=8.2 Hz, 1 H, 5′-H), 7.75 (d, J=9.0 Hz, 1 H, 3-H), 7.53-7.45 (m, 2 H, Ar) overlapped by 7.50 (d, J=9.2 Hz, 1 H, 3′-H), 7.28 (ddd, J=8.2, 6.8, 1.2 Hz, 1 H, 6 or 6′ or 7 or 7′-H), 7.21 (ddd, J=8.4, 6.8, 1.2 Hz, 1 H, 6 or 6′ or 7 or 7′-H), 7.07 (app. d, J=8.4 Hz, 2 H, Ts-o), 7.05 (br d, J=8.4 Hz, 1 H, 8 or 8′-H), 6.97 (br d, J=8.4 Hz, 1 H, 8 or 8′-H), 6.73 (app. d, J=8.0 Hz, 2 H, Ts-m). ¹³C NMR (100.6 MHz, CDCl₃): δ=146.0, 145.5, 144.4, 133.2, 133.0. 132.8, 132.0, 131.8, 131.2, 130.9, 129.1 (2 C, Ts), 128.2, 127.9, 127.5, 127.25, 127.2, 127.1 (2 C, Ts), 126.6, 126.5, 126.3, 124.7, 122.2, 121.4, 119.4, 21.6 (Me), CF₂ and CF₃ groups not apparent due to extensive coupling. ¹⁹F NMR (282.4 MHz, CDCl₃): δ=−81.2 (m, CF₃), −110.6 (m, CF₂), −121.6 (br, CF₂), −126.4 (m, CF₂). [HRMS (FAB): found: 722.0462. Calcd. for C₃₁H₁₉F₉O₆S₂ 722.0479].

Selected data for (35): ¹H NMR (CDCl₃): δ=7.45 (ddd, J=8.8, 7.1, 1.8 Hz, 1 H, Ar of biphenyl), 7.43-7.39 (m, 2 H, Ar of biphenyl), 7.38 (ddd, J=7.6, 7.2, 1.5 Hz, 1 H, Ar of biphenyl), 7.32 (app. d, J=8.3 Hz, 2 H, C₆H₄Me), 7.29-7.25 (m, 3 H, Ar of biphenyl), 7.11 (app. d, J=8.3 Hz, 2 H, C₆H₄Me), 7.06 (dd, J=8.0, 1.7 Hz, 1 H, Ar of biphenyl). ¹³C NMR (100.6 MHz, CDCl₃): δ=146.8 (C2), 145.0 (C2′), 132.7 (C), 132.4 (CH), 131.8 (CH), 130.7 (C), 130.2 (CH), 129.6 (2 C, Ts-o), 129.5 (CH), 129.4 (C), 128.0 (2 C, Ts-m), 127.9 (CH), 127.2 (CH), 123.1 (CH), 121.2 (CH), 21.7 (Me).

Additional compound prepared by this route. (31) Cₙ=1,1′-binaphthyl, R²⁵=Ph, R^{perF}=CF₃ ¹H NMR (400.1 MHz, CDCl3): δ=8.07 (d, J=9.0 Hz, 1 H, 4-H of binaphthyl), 7.95 (d, J=8.5 Hz, 1 H, 5-H of binaphthyl) overlapped by 7.95 (d, J 9.3 Hz, 1 H, 4′-H of binaphthyl), 7.88 (d, J=8.2 Hz, 1 H, 5′-H of binaphthyl), 7.74 (d, J 9.0 Hz, 1 H, 3-H of binaphthyl), 7.51 (ddd, J=8.0, 5.3, 1.2 Hz, 1 H, 7 or 7′-H of binaphthyl), 7.49 (ddd, J=8.2, 5.3, 1.2 Hz, 1 H, 7 or 7′-H of binaphthyl), 7.45 (d, J=9.1 Hz, 1 H, 3′-H of binaphthyl), 7.31-7.21 (m, 5 H, 6,6′ and 8-H of binaphthyl and Ph-o), 7.09-6.98 (m, 4 H, 8′-H of binaphthyl and Ph-m+p). Demonstration of chemospecificity on initial coupling step, preparation of (34a): In the preparation of (33a) [from (Sₐₓ)-Binol (1.00 g, 3.52 mmol) the presence of intermediate (34a) could be detected (R_{f} 0.45, CH₂Cl₂). After 8 h quantitative yields were realised (1.55 g, 3.52 mmol, 100%). M.p. 61° C. [α]_{D}²⁵-46 (c=1.00, CHCl₃). Compound (2) could be crystallised as colourless plates of a toluene solvate, C₂₇H₂₀O₄S CH₃C₆H₅: calcd. C 76.7, H 5.3; found C 76.7, H 5.3%. ¹H NMR (400.1 MHz, CDCl₃) of the toluene mono-solvate compound: δ=8.10 (d, J=9.0 Hz, 1 H, 4′-H), 8.0 (d, J=8.2 Hz, 1 H, 5′-H), 7.9 (d, J=9.0 Hz, 1 H, 4-H), 7.8 (d, J=8.2 Hz, 1 H, 5-H), 7.7 (d, J=9.0 Hz, 1 H, 3′-H), 7.5 (app. t, J=7.1 Hz, 1 H, 7 or 7′ or 6 or 6′-H), 7.37 (app. t, J=7.0 Hz, 1 H, 7 or 7′ or 6 or 6′-H), 7.3-7.1 (m, 11H, Ar), 6.9 (d, J=8.3 Hz, 2 H, Ts-m), 6.8 (d, J=8.5 Hz, 1 H, 8 or 8′-H), 5.16 (s, 1H, OH), 2.4 (s, 3H, Me toluene), 2.3 (s, 3H, Me (2)). ¹³C NMR (100.6 MHz, CDCl₃) of pure, toluene freed (34a): δ=151.6, 146.4, 144.7, 133.4 (2C), 132.7, 132.5, 130.9, 130.4, 129.3(2C), 128.8, 128.3, 127.8, 127.6, 127.5 (2 C), 126.7, 126.6, 126.2, 124.6, 123.8, 123.3, 121.7, 118.1, 113.7, 21.6 (CH₃ Ts).

Preparation of (36a): To a solution of (Sₐ)-(33a) in 30 mL of freshly distilled THF (1.95 g, 3.4 mmoL), was added drop-wise a solution of MeMgBr in Et₂O (10.2 mmoL) at 0° C. The reaction mixture was allowed to come to ambient temperature and then stirred under reflux (bath temperature 80° C., 16-20 h). Completion of the coupling was indicated by the reaction becoming deep orange-brown from its original lemon yellow colour and by TLC analysis (9:1 light petroleum:EtOAc; R_{f} 4 0.32; R_{f} 3 0.15). Unreacted MeMgBr was quenched with methanol (25 mL). An aqueous solution of KOH (3.8 g in 25 mL of H₂O) was added and the reaction mixture stirred at 80° C. for 12 hours. The reaction mixture was acidified with conc. HCl and extracted with CH₂Cl₂. The organic layers were dried over MgSO₄ and the solvent removed by vacuum. The crude product was purified by chromatography (Et₂O:light petroleum=1:5) Rf (3)=0.33. Compound (3) was obtained as a colourless solid (0.54 g, 1.91 mmoL, 56% isolated yield). [α]_{D}²⁵+87 (c=1.00, CHCl₃). ¹H NMR (400.1 MHz, CDCl₃): δ=7.95 (d, J=8.5 Hz, 1 H, 4-H), 7.91 (app. d, J=8.8 Hz, 2 H, 4′-H overlapped by 5-H), 7.87 (d, J=8.1 Hz, 1 H, 5′-H), 7.56 (d, J=8.5 Hz, 1 H, 3-H), 7.45 (ddd, J=1.3, 6.7, 8.1, 1 H, 6 or 6′ or 7 or 7′-H), 7.35 (d, J=8.8 Hz, 1 H, 3′-H) overlapped by 7.34-7.20 (m, 4 H, 6 or 6′ or 7 or 7′ and 8 or 8′-H), 6.98 (d, J=8.4 Hz, 1 H, 8 or 8′-H), 4.77 (s, 1 H, OH), 2.15 (s, 3 H, Me). ¹³C NMR (100.6, CDCl₃): δ=150.67, 137.18, 133.26, 133.17, 132.52, 129.77, 129.11, 129.01, 128.98, 128.62, 128.14, 128.10, 126.83, 126.67, 125.52, 125.39, 124.49, 123.40, 117.54, 117.35, 20.14. These values are concordant with those published data for an alternative route [Y. Tamai, T. Nakano, S. Miyano, J. Chem. Soc., Perkin Trans. 1 1994, 439-445.].

Similarly prepared were (36b) and (36c). Selected data below.

For (36b): ¹H NMR (400.1 MHz, CDCl₃): δ=7.99 (d, J=8.5 Hz, 1 H, 4-H), 7.92 (app. d, J=8.7 Hz, 2 H, 4′-H overlapped by 5-H), 7.87 (d, J=8.1 Hz, 1 H, 5′-H), 7.61 (d, J=8.5 Hz, 1 H, 3-H), 7.45 (ddd, J=1.2, 6.7, 8.1, Hz, 1 H, 6 or 6′-H), 7.35 (d, J=8.8 Hz, 1 H, 3′-H) 7.32 (ddd, J=1.2, 6.8, 8.1 Hz, 1 H, 6 or 6′-H), 7.27 (ddd, J=1.3, 6.8, 8.5 Hz, 1 H, 7 or 7′-H), 7.22 (ddd, J=1.3,6.8,8.5Hz, 1 H, 7 or7′-H), 7.18 (d, J=8.5Hz, 1 H, 8 or 8′-H), 6.98 (d, J=8.5 Hz, 1 H, 8 or 8′-H), 4.78 (s, 1 H; OH), 2.47 (q, J=7.6 Hz, 2 H, CH₂Me), 1.07 (t, J=7.6 Hz, 3 H, CH₂Me). ¹³C NMR (100.6 MHz, CDCl₃): δ=151.0 (C), 143.3 (C), 133.8 (C), 133.2 (C), 132.6 (C), 129.8 (CH), 129.4 (CH), 129.1 (C), 128.1 (2 C, CH), 127.9 (C), 127.5 (CH), 126.8 (CH), 126.6 (CH), 125.7 (CH), 124.8 (CH), 123.4 (CH), 117.4 (C), 117.3 (CH), 27.0 (CH₂), 15.3 (Me).

Representative lithiation of (36a) and its reaction with phosphorus electrophiles. A solution of nBuLi (1.2 mL of 2.5 M hexane solution, 3.0 mmol) and TMEDA (0.35 g, 3.00 mmol) were added to a solution of (36a) (0.284 g, 1.00 mmol) in diethylether (10 mL) under argon at 0° C. [Lithiation was evidenced by the formation of a copious red precipitate and by test quenching of the anion with D₂O. The presence of an isotopically shifted 1:1:1 triplet confirmed the presence of >90% CH₂D after 24 h]. Alternatively, the anion could be prepared by treatment of (36a) with tBuOK/nBuLi at −40° C. The anion was either treated directly with PCl₃, Cl₂PPh, Cl₂PN(CHMePh)₂, P(OPh)₃, PhP(OR)₂ or (RO)₂PN(CHMePh)₂ [R=Et, Ph] to afford mixtures containing, by ³¹P NMR the desired ligands.

Selected data for 12 R¹¹=OPh below.

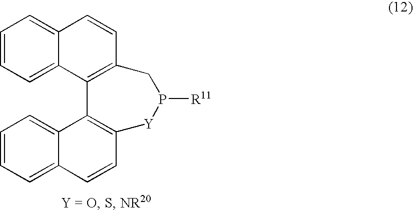

¹H NMR (400.1 MHz, CDCl₃): δ=7.98-7.86 (m, 4 H, 2×4-H and 5-H binaphthyl), 7.76-6.88 (m, 13H, Ar), 6.74-6.68 (m, 2 H, 2×H8 binaphthyl), 2.18-2.10 (m, 2 H, CH₂); ³¹P NMR (202.5 MHz, C₆D₆): δ=137.8, 137.4. [HRMS (+ES on M+H₂O adduct): found: 424.1222. Calcd. for C₂₇H₂₁O₃P 424.1228]. The compound existed as a mixture of P-epimers.

### Representative Preparation of P-Chiral Type 8 Ligand Wherein X and Y are O and NR²⁰.

This ligand was prepared from (35) by the three stage sequence shown above and in the following Scheme

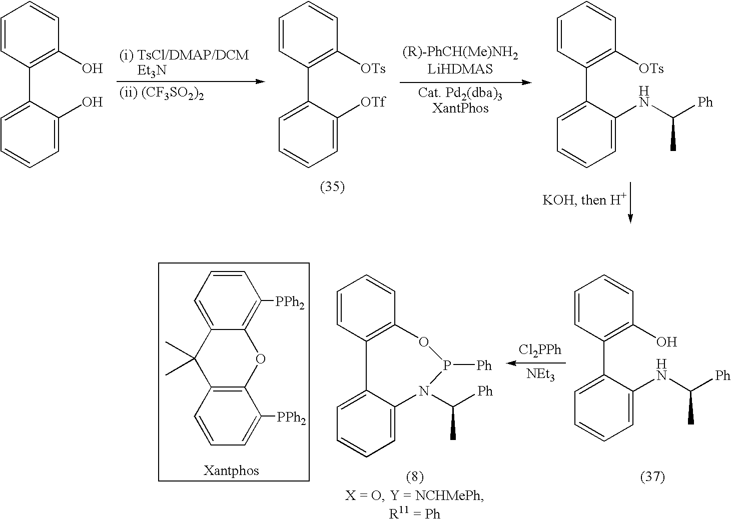

Preparation of (37): A solution of LiHDMS (6.2 mL of 1.5 M THF solution, 9.3 mmol) was added, under an argon atmosphere to a mixture of (35) (2.00 g, 4.23 mmol), Pd₂(dba)₃ (39 mg, 0.04 mmol, 1 mol %), Xantphos (59 mg, 0.1 mmol, 2.4 mol %) and excess (R)-PhCH(Me)NH₂ (2.3 mL). The resulting dark brown reaction solution was heated to 65° C. (8-16 h), the excess liquids removed under vacuum and the residue chromatographed on silica using 1:1 hexane:dichloromethane collecting the fraction with Rf=0.46 which corresponded to the intermediate tosylated amine in 56% yield [¹H NMR (400.1 MHz, CDCl₃): δ=7.50-7.25 (m, 14 H, Ar), 7.13-7.09 (m, 2 H, Ts-m), 7.07 (dd, J=7.9, 1.9 Hz, Ar), 5.12 (br d, J ˜7 Hz, exchanges with D₂O, NH), 4.82 (quintet, J=7.2 Hz, 1 H, CHMe), 2.43 (s, 3 H, C₆H₄Me), 1.67 (d, J=7.2 Hz, 3 H, CHMe).] Hydrolysis with excess KOH followed by careful neutralisation afforded (37).

Ligand (8, X=O, Y=NCHMePh, R¹¹=Ph) was prepared by the reaction of (37) with Cl₂PPh in the presence of NEt₃.

General reaction conditions for the handling of DABAL reagents and the catalytic reaction. Reactions were carried out under argon in THF (laboratory reagent grade, Fischer Scientific) distilled from Na-benzophenone (except in one reaction of PhCHO which was conducted in the THF as supplied). DABAL-Me₃ 1-Me₃ could be handled in air for 30 mins ˜4 hours without appreciable decomposition depending on ambient conditions. DABAL-Et₃ 1-Et₃ was prepared by addition of neat AlEt₃ (2 equiv.) to DABCO in THF; both it and AlR₃ were handled only as ca. 2.0 M solutions. Other DABAL reagents were handled in a similar manner to DABAL-Et₃ 1-Et₃. All of the other reaction components are commercially available (STREM or Sigma-Aldrich). The Ni(acac)₂ used was the anhydrous (95% grade); organoaluminium reagents were used neat or as 2.0 M hexanes solutions. All the secondary alcohols (PA1) are literature compounds that were identified by comparison of their ¹H NMR spectra with genuine samples measured at ambient temperature (nominally 22° C.) referenced to TMS. Primary stereochemical correlations relied on the polarimetry data of Seebach [D. Seebach, A. K. Beck, R. Imwinkelried, S. Roggo, A. Wonnacott, Helv. Chim. Acta 1987, 70, 954-974] and genuine scalemic GC samples of (PA1) from our own group's earlier studies supported by other polarmetric literature studies. All ee determinations were by chiral GC under conditions similar to those described previously by us. [A. J. Blake, A. Cunningham, A. Ford, S. J. Teat, S. Woodward, Chem. Eur. J. 2000, 6, 3586-3594].

The process of the invention is now described with reference to the following Scheme

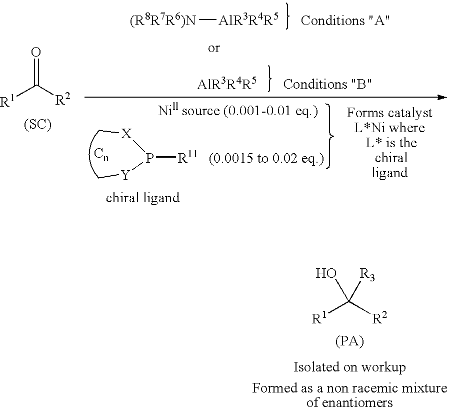

R¹-R⁸=H, Alkyl, alkenyl, allyl, akynyl, aryl, heteroaryl and appropriately substituted versions thereof.

R¹¹=R¹ and H, OH OR, NHR, NR₂ where “R” is an appropriately substituted member of group R¹

X,Y=O, S, CH₂ Cₙ is a chiral aliphatic or aromatic tether, typically (but not exclusively) based
CHR, CR₂ on 1,1-binaphthyl.
NR

Representative nickel-catalysed additions to prochiral aldehydes using DABAL reagents (Conditions A). Under an argon atmosphere, Ni(acac)₂ (0.6 mg, 2.33 μmol, 1 mol %) and (RₐₓS,S)-L1 (2.7 mg, 0.005 mmol, 2 mol %) were stirred in dry THF (2 ml) at 5° C. for 10-30 min. Neat aldehyde (0.25 mmol) was added and after 10 min DABAL-Me₃ 1 (84 mg, 0.325 mmol, 1.3 eq.) was added. The straw yellow reaction mixture was stirred (1-3 h) before being quenched with NH₄Cl(aq). Yields of the sec-alcohol PA1 or PA2 were attained either by isolation or by GC assay after addition of a dodecane internal standard.

Representative nickel-catalysed addition to prochiral aldehydes using AlR₃ species (Conditions B). Under an argon atmosphere, Ni(acac)₂ (0.6 mg, 2.33 μmol, 1 mol %) and (Rₐₓ,S,S)-L1 (2.7 mg, 0.005 mmol, 2 mol %) were stirred in dry THF (2 ml) at ambient temperature for 10-20 min. The solution was cooled to −20° C. and neat aldehyde (0.25 mmol) was added. After 5 min AlR₃ (R=Me, Et) (0.25 mL of 2.0 M hexanes solution, 0.5 mmol, 2.0 eq.) was added. The straw yellow reaction mixture was stirred (6 h) before being quenched with 2M aqueous HCl. Yields of the sec-alcohol PA1 (from Me addition) or PA2 (from Et addition) were attained either by isolation or by GC assay after addition of a dodecane internal standard.

Specific substrate examples are given in Tables 1-2, supported by the data on the following pages.

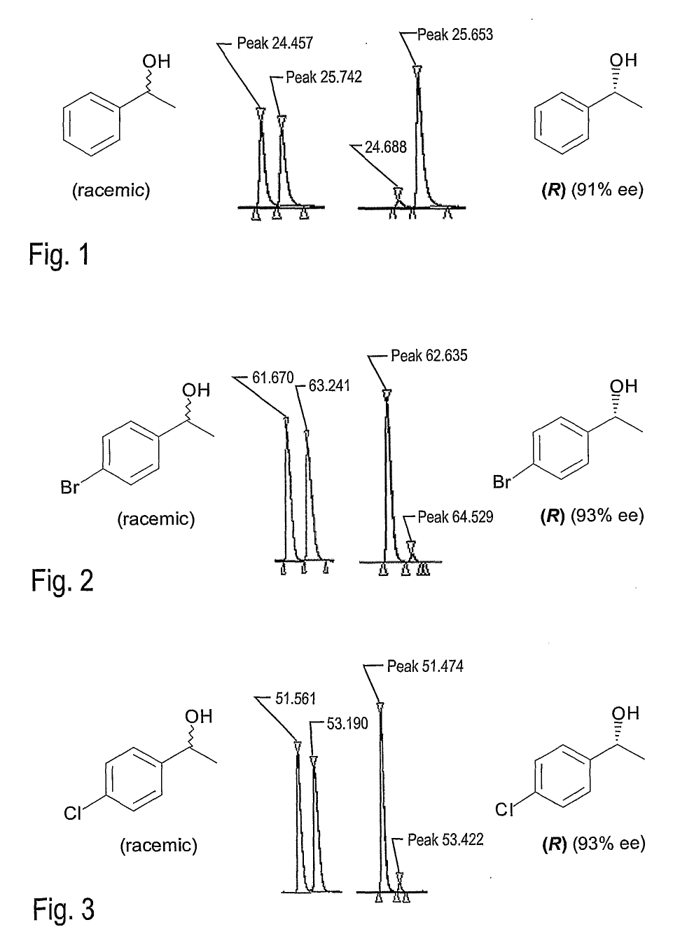

(+)—(R)—PhCH(Me)OH PA1a. ¹H NMR (500.1 MHz, CDCl₃) δ=1.57 (d, J=6.4 Hz, 3 H; CH₃), 2.36 (s, br, 1 H; OH), 4.95 (q, J=6.4 Hz, 1 H; CH), 7.20-7.30 (m, 1H; Ar), 7.36-7.46 (m, 4 H; Ar). Determination of enantiomeric excess: GC(Lipodex A; 75° C. isothermal) (S)-PA1a (24.6 min), (R)-PA1a (25.7 min). GC traces of genuine racemic and chiral catalysis derived PA1a samples are shown in FIG. 1.

(+)—(R)-(4-BrPh)CH(Me)OH PA1b. ¹H NMR (500.1 MHz, CDCl₃) δ=1.50 (d, J=6.5 Hz, 3 H; CH₃), 2.65 (s, br, 1 H; OH), 4.87 (q, J=6.5 Hz, 1 H; CH), 7.28 (app d, J=8.5 Hz, 2 H; Ar), 7.52 (app d, J=8.5, 2 H; Ar). Second order fine structure is associated with the doublets at 7.28 and 7.52. GC(Cyclodex-B, initially 100° C. ramped to 150° C. at 1° C. min⁻¹): (R)-PA1b (62 min), (S)-PA1b (63 min). GC traces of genuine racemic and chiral catalysis derived PA1b samples are shown in FIG. 2.

(+)—(R)-(4-CIPh)CH(Me)OH PA1c. ¹H NMR (500.1 MHz, CDCl₃) δ=1.48 (d, J=6.5 Hz, 3 H; CH₃), 3.09 (s, br, 1 H; OH), 4.85 (q, J=6.5 Hz, 1 H; CH), 7.31 (d, J=8.4 Hz, 2 H; Ar), 7.34 (d, J=8.4 Hz, 2 H; Ar). GC(Cyclodex-B, initially 100° C. ramped to 150° C. at 1° C. min⁻¹): (R)-PA1c (51.5 min), (S)-PA1c (53.4 min). GC traces of genuine racemic and chiral catalysis derived PA1c samples are shown in FIG. 3.

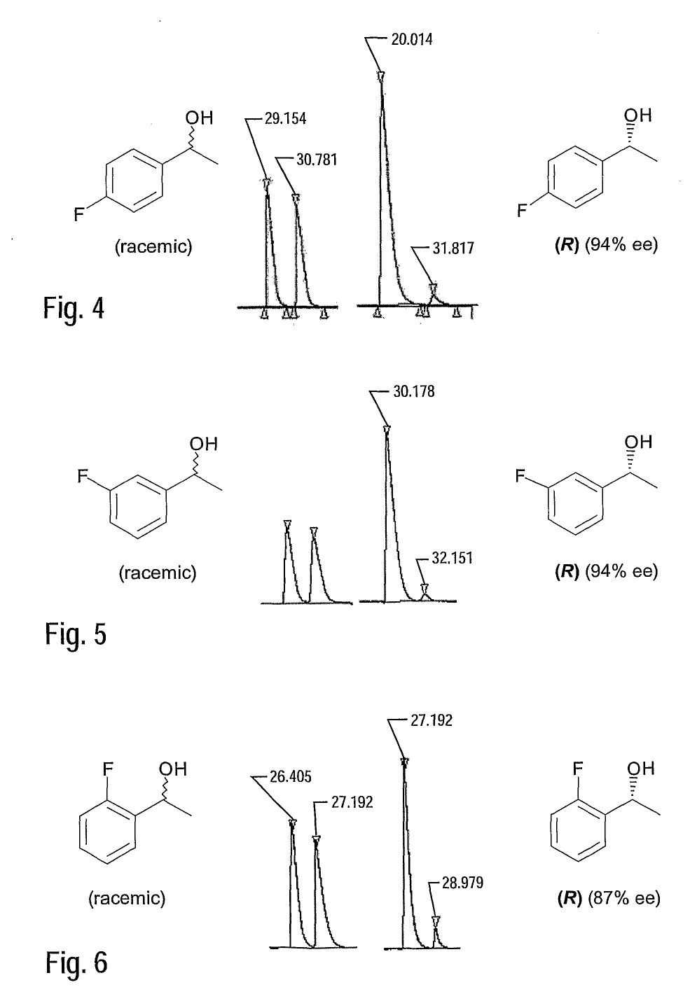

(+)—(R)-(4-FPh)CH(Me)OH PA1d. ¹H NMR (500.1 MHz, CDCl₃) δ=1.52 (d, J=6.4 Hz, 3 H; CH₃), 2.43 (s, br, 1 H; OH), 4.92 (q, J=6.4 Hz, 1 H; CH), 7.09 (app t, J=8.7 Hz, 2 H; Ar), 7.39 (app t, J=5.5 Hz, 2 H; Ar). GC(Cyclodex-B, initially 100° C. ramped to 150° C. at 1° C. min⁻¹): (R)-PA1d (29 min), (S)-PA1d (31 min). GC traces of genuine racemic and chiral catalysis derived PA1d samples are shown in FIG. 4.

(+)—(R)-(3-FPh)CH(Me)OH PA1e. ¹H NMR (500.1 MHz, CDCl₃) δ=1.1.49 (d, J=6.5 Hz, 3 H; CH₃), 3.08 (s, br, 1 H; OH), 4.88 (q, J=6.5 Hz, 1 H; CH), 6.96-6.87 (m, 1 H; Ar), 7.10-7.17 (m, 2 H; Ar), 7.32-7.36 (m, 1 H; Ar). GC(Cyclodex-B, initially 100° C. ramped to 150° C. at 1° C. min⁻¹): (R)-PA1e (30 min), (S)-PA1e (32 min). GC traces of genuine racemic and chiral catalysis derived PA1e samples are shown in FIG. 5.

(+)—(R)-(2-FPh)CH(Me)OH PA1f. ¹H NMR (500.1 MHz, CDCl₃) δ=1.57 (d, J=6.5 Hz, 3 H; CH₃), 2.47 (s, br, 1 H; OH), 5.26 (q, J=6.6 Hz, 1 H; CH), 7.09 (ddd, J=8.2, 7.6, 1.2, 1 H; Ar), 7.22 (td, J=7.6, 1.2, 1 H; Ar), 7.29-7.37 (m, 1 H; Ar), 7.55 (td, J=7.6, 1.8, 1 H; Ar). GC(Cyclodex-B, initially 100° C. ramped to 150° C. at 1° C. min⁻¹): (R)-PA1f (26.5 min), (S)-PA1f (28.5 min). GC traces of genuine racemic and chiral catalysis derived PA1f samples are shown in FIG. 6.

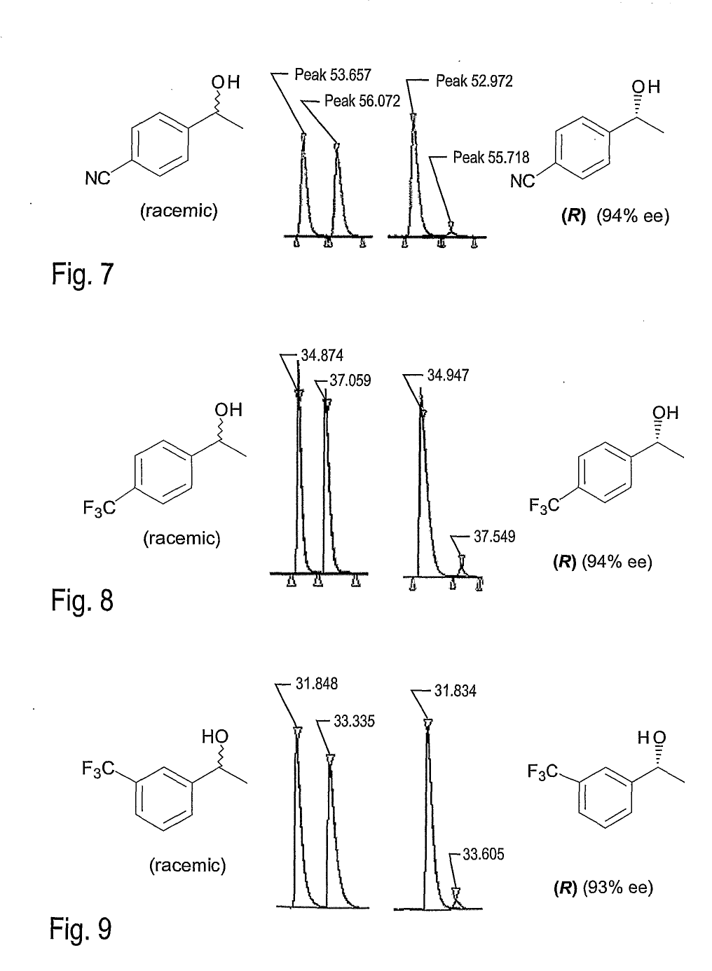

(+)—(R)-[(4-CN)Ph]CH(Me)OH PA1g. ¹H NMR (400.1 MHz, CDCl₃) δ=1.46 (d, J=6.4 Hz, 3 H; CH₃), 2.98 (s, br, 1 H; OH), 4.92 (q, J=6.4 Hz, 1 H; CH), 7.46 (app d, J=8.6 Hz, 2 H; Ar), 7.58 (app d, J=8.6 Hz, 2 H; Ar). Second order fine structure is associated with the doublets at 7.46 and 7.58. GC(Cyclodex-B, initially 100° C. ramped to 150° C. at 1° C. min⁻¹): (R)-PA1g (53 min), (S)-PA1g (56 min). GC traces of genuine racemic and chiral catalysis derived PA1g samples are shown in FIG. 7.

(+)—(R)-[(4-CF₃)Ph]CH(Me)OH PA1h. ¹H NMR (400.1 MHz, CDCl₃) δ=1.41 (d, J=6.4 Hz, 3 H; CH₃), 1.98 (s, br, 1 H; OH), 4.87 (q, J=6.4 Hz, 1 H; CH), 7.39 (d, J=8.0 Hz, 2 H; Ar), 7.52 (d, J=8.2 Hz, 2 H, Ar). GC(Cyclodex-B, initially 100° C. ramped to 150° C. at 1° C. min⁻¹): (R)-PA1h (35 min), (S)-PA1h (37 min). GC traces of genuine racemic and chiral catalysis derived PA1h samples are shown in FIG. 8.

(+)—(R)-[(3-CF₃)Ph]CH(Me)OH PA1i. ¹H NMR (500.1 MHz, CDCl₃) δ=1.54 (d, J=6.5 Hz, 3 H; CH₃), 2.83 (s, br, 1 H; OH), 4.97 (q, J=6.5 Hz, 1 H; CH), 7.52 (t, J=7.7 Hz, 1 H; Ar), 7.57-7.60 (m, 2 H; Ar), 7.69 (s, br, 1 H; Ar). GC(Cyclodex-B, initially 100° C. ramped to 150° C. at 1° C. min⁻¹): (R)-PA1i (35 min), (S)-PA1i (37 min). GC traces of genuine racemic and chiral catalysis derived PA1i samples are shown in FIG. 9.

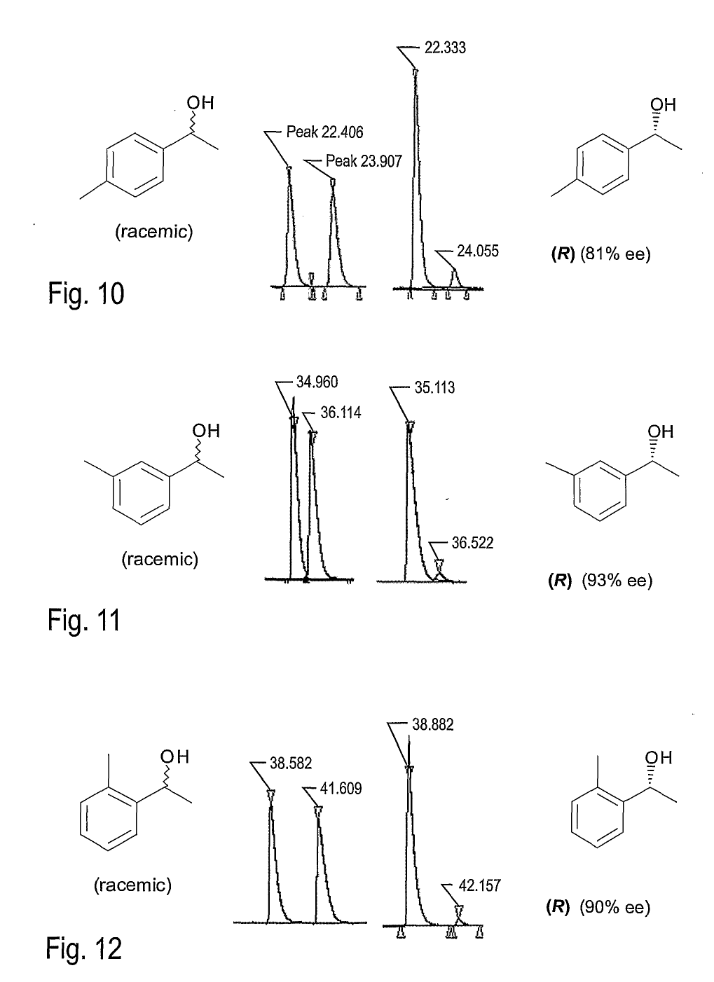

(+)—(R)-4-MePhCH(Me)OH PA1j.¹H NMR (500.1 MHz CDCl₃) δ=1.57 (d, J=6.5 Hz, 3 H; CH₃), 1.89 (br, s, 1 H; OH), 2.44 (s, 3 H; ArCH₃), 4.95 (q, J=6.5 Hz, 1 H; CH), 7.26 (d, J=8.0 Hz, 2 H; Ar), 7.36 (d, J=8.0, Hz, 2 H; Ar). GC(Cyclodex-B, initially 100° C. ramped to 150° C. at 1° C. min⁻¹): (R)-PA1j (22.4 min), (S)-PA1j (24.0 min). GC traces of genuine racemic and chiral catalysis derived PA1j samples are shown in FIG. 10.

(+)—(R)-3-MePhCH(Me)OH PA1k. ¹H NMR (400.1 MHz, CDCl₃) δ=1.38 (d, J=6.4 Hz, 3 H; CH₃), 2.00 (br, s, 1 H; OH), 2.27 (s, 3 H; ArCH₃), 4.74 (q, J=6.4 Hz, 1 H; CH), 6.99 (d, J=7.6 Hz, 1 H; Ar), 7.05-7.09 (m, 2 H; Ar), 7.13-7.16 (m, 1 H; Ar). GC(Cyclodex-B, initially 100° C. ramped to 150° C. at 1° C. min⁻¹): (R)-PA1k (22.4 min), (S)-PA1k (24.0 min). GC traces of genuine racemic and chiral catalysis derived PA1k samples are shown in FIG. 11.

(+)—(R)-2-MePhCH(Me)OH PA1l. ¹H NMR (400.1 MHz, CDCl₃) δ=1.34 (d, J=6.4 Hz, 3 H; CH₃), 1.98 (br, s, 1 H; OH), 2.24 (s, 3 H; ArCH₃), 4.99 (q, J=6.4 Hz, 1 H; CH), 7.02-7.09 (m, 2 H; Ar), 7.11-7.15 (m, 1 H; Ar), 7.40 (d, J=8.4 Hz, 1 H; Ar). GC(Cyclodex-B, initially 100° C. ramped to 150° C. at 1° C. min⁻¹): (R)-PA1l(39 min), (S)-PA1l (42 min). GC traces of genuine racemic and chiral catalysis derived PA1l samples are shown in FIG. 12.

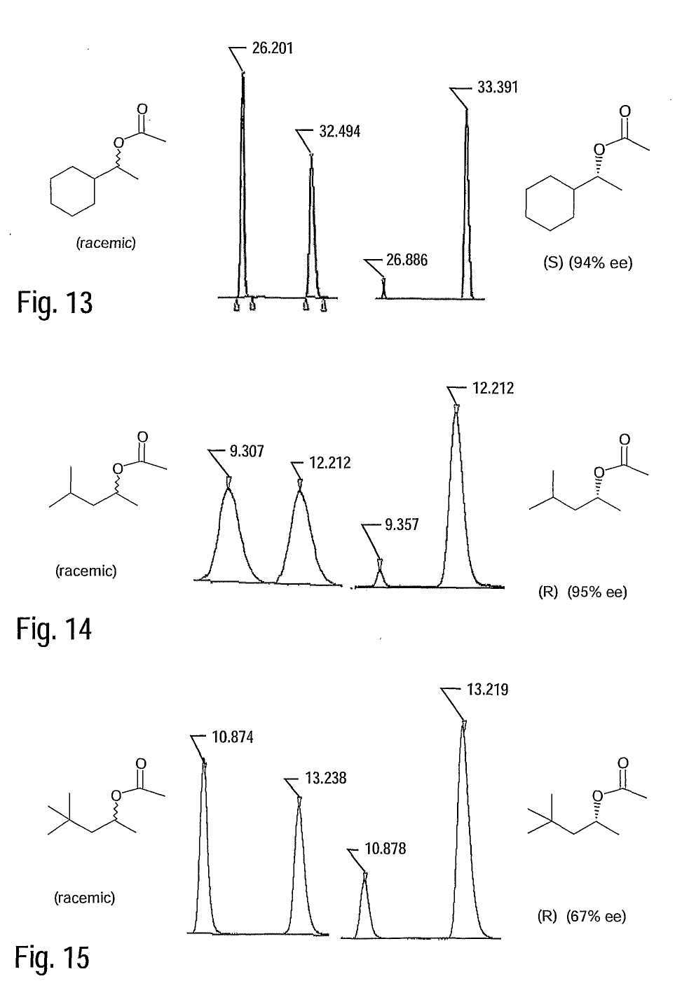

(−)—(R)—(c—C₆H₁₁)CH(Me)OH PA1m. ¹H NMR (400.1 MHz, CDCl₃) δ=1.17-1.22 (m, 6 H; cC₆H₁,) overlapping 1.21 (d, J=6.4 Hz GC, 3 H; CH₃), 1.59-1.82 (m, 5 H; cC₆H₁₁), 2.33 (s, br, 1 H; OH), 3.46 (q, J=6.4 Hz, 1 H; CH). GC(2,6-me-3-pe-γ-CD, 60° C. initially for 10 min, then ramped to 90° C. at 5° C. min⁻¹ using acetate of PA1m): (S)-PA1m-OAc (26 min), (R)-PA1m-OAc (33 min). GC traces of genuine racemic and chiral catalysis derived PA1m samples (acetates) are shown in FIG. 13.

(−)—(R)-i-BuCH(Me)OH PA1n. ¹H NMR (500.1 MHz, CDCl₃) δ=0.85 (d J=6.6 Hz, 1 H; CHCH_{3α}), 0.86 (d J=6.6 Hz, 1 H; CHCH_{3β}), 1.11 (d, J=6.1 Hz, 3 H; HOCHCH₃), 1.12-1.17 (m, 1 H; CH_{2α}-i-Pr), 1.34 (ddd J=14.1, 8.1, 6.1 Hz, 1 H; CH_{2β}-i-Pr), 2.52 (s, br, 1 H; OH), 3.77-3.83 (m, 1 H; CHOH). GC(6-me-2,3-pe-γ-CD, 50° C. isothermal using acetate of PA1n): (S)-PA1n-OAc (9.3 min), (R)-PA1n-OAc (12.2 min). GC traces of genuine racemic and chiral catalysis derived PA1n samples (acetates) are shown in FIG. 14.

(−)—(R)-t-BuCH₂CH(Me)OH PA1o. ¹H NMR (500.1 MHz, CDCl₃) δ=0.83 (s, 9 H; t-Bu), 1.07 (d, J=6.2 Hz, 3 H; CHCH₃), 1.21 (dd J=13.4, 3.2 Hz, 1 H; CH_{2α}-t-Bu), 1.28 (dd J=13.4, 7.8 Hz, 1 H; CH_{2β}-t-Bu), 2.08 (s, br, 1 H; OH), 3.86 (ddq, J=7.8, 3.0, 6.2 Hz, 1 H; CHOH). GC(6-me-2,3-pe-γ-CD, 50° C. isothermal using acetate of PA1o): (S)-PA1o-OAc (10.9 min), (R)-PA1O-OAc (13.2 min). GC traces of genuine racemic and chiral catalysis derived PA1p samples (acetates) are shown in FIG. 15.

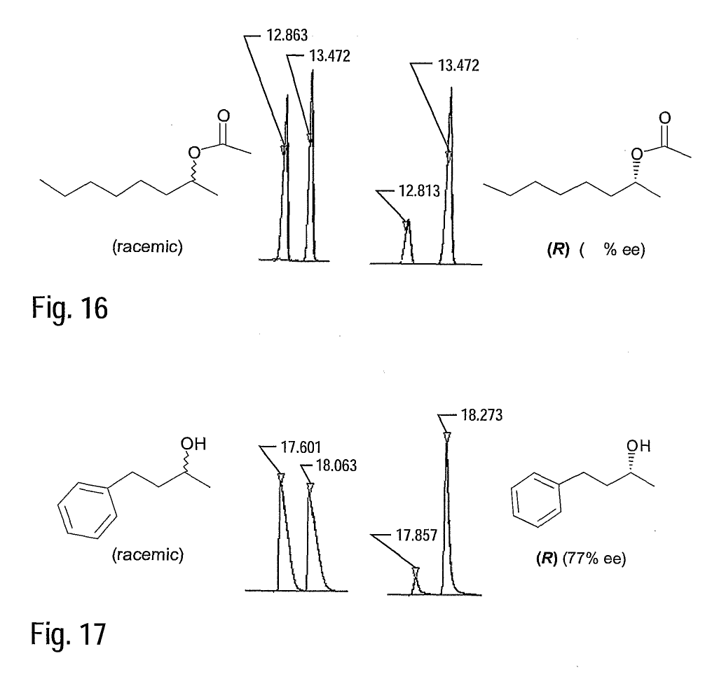

(−)—(R)-n-C₆H₁₁CH(Me)OH PA1p. ¹H NMR (500.1 MHz CDCl₃) δ=0.89 (t, J=6.8 Hz, 3 H; (CH₂)₅CH₃), 1.21 (d, J=6.2 Hz, 3 H; CHCH₃), 1.27-1.52 (m, 10 H; (CH₂)₅), 2.80 (s, br, 1 H; OH), 3.80-3.84 (m, 1 H; CHOH). GC(6-me-2,3-pe-γ-CD, 70° C. initially for 5 min, then ramped to 160° C. at 2° C. min⁻¹ using acetate of PA1p): (S)-PA1p-OAc (12.8 min), (R)-PA1p-OAc (13.4 min). GC traces of genuine racemic and chiral catalysis derived PA1p samples (acetates) are shown in FIG. 16.

(−)—(R)—PhCH₂CH₂CH(Me)OH PA1q. ¹H NMR (500.1 MHz CDCl₃) δ=1.27 (d, J=6.0 Hz, 3 H; CH₃), 1.75-1.88 (m, 2 H; CH₂Ph), 2.16 (s, br, 1 H; OH), 2.71 (ddd, J=7.0, 9.5, 14.0 Hz, 1 H; CH_{2α}CH), 2.80 (ddd, J=6.0, 9.4, 14.0 Hz, 1 H; CH_{2β}CH), 7.21-7.29 (m, 3 H; m+p-Ph), 7.32-7.35 (m, 2 H; o-Ph). GC(2,6-me-3-pe-γ-CD, 110° C. initially for 5 min, then ramped to 180° C. at 5° C. min-¹): (S)-PA1q (17.5 min), (R)-PA1q (18 min). GC traces of genuine racemic and chiral catalysis derived PA1q samples are shown in FIG. 17.

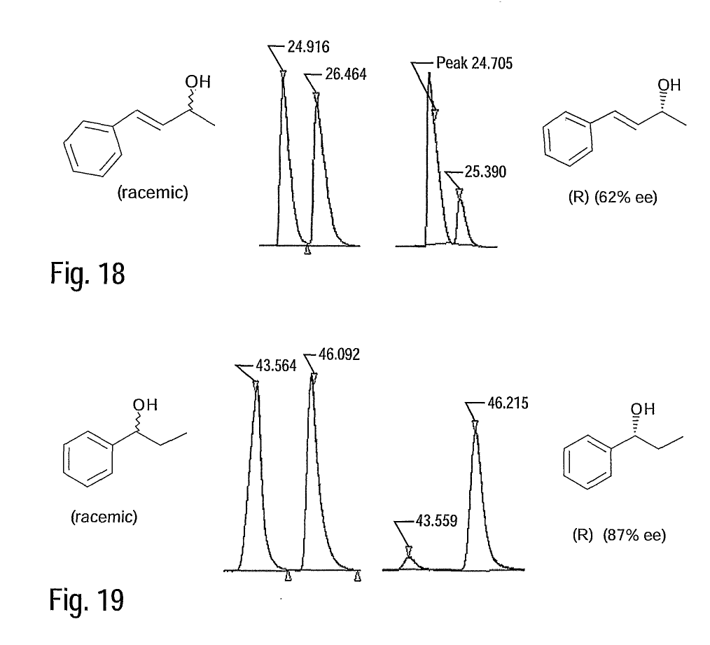

(+)—(R)—(E)—PhCH=CHCH(Me)OH PA1r. ¹H NMR (400.1 MHz, CDCl₃) δ=1.30 (d, J=6.4 Hz, 3 H; CH₃), 1.56 (s, br, 1 H; OH), 4.42 (quintet, J=6.4 Hz, 1 H; CHOH), 6.21 (dd, J=16.0, 6.4 Hz, 1H; =CHCH), 6.50 (d, J=16.0 Hz, 1 H; =CHPh), 7.14-7.18 (m, 1 H; p-Ph), 7.20-7.26 (m, 2 H; m-Ph), 7.30-7.33 (m, 2 H; o-Ph). GC(2,6-me-3-pe-γ-CD, initially 120° C. ramped to 150° C. at 1° C. min⁻¹): (R)-4s (24.5 min), (S)-4s (25 min). GC traces of genuine racemic and chiral catalysis derived 4s samples are shown in FIG. 18.

(+)—(R)—PhCH(Et)OH PA2a. ¹H NMR (500.1 MHz, CDCl₃) δ=0.99 (t, J=7.4 Hz, 3 H; CH₂CH₃), 1.77-1.93 (m, 2 H; CH₂CH₃), 2.67 (d J=3.4 Hz, 1 H; OH), 4.62 (dt, J=3.4, 6.6 Hz, 1 H; CHOH), 7.21-7.44 (m, 5H; Ph). GC(Lipodex-A, 75° C. isothermal): (S)-PA2a (43.6 min), (R)-PA2a (46.2 min). GC traces of genuine racemic and chiral catalysis derived PA2a samples are shown in FIG. 19.

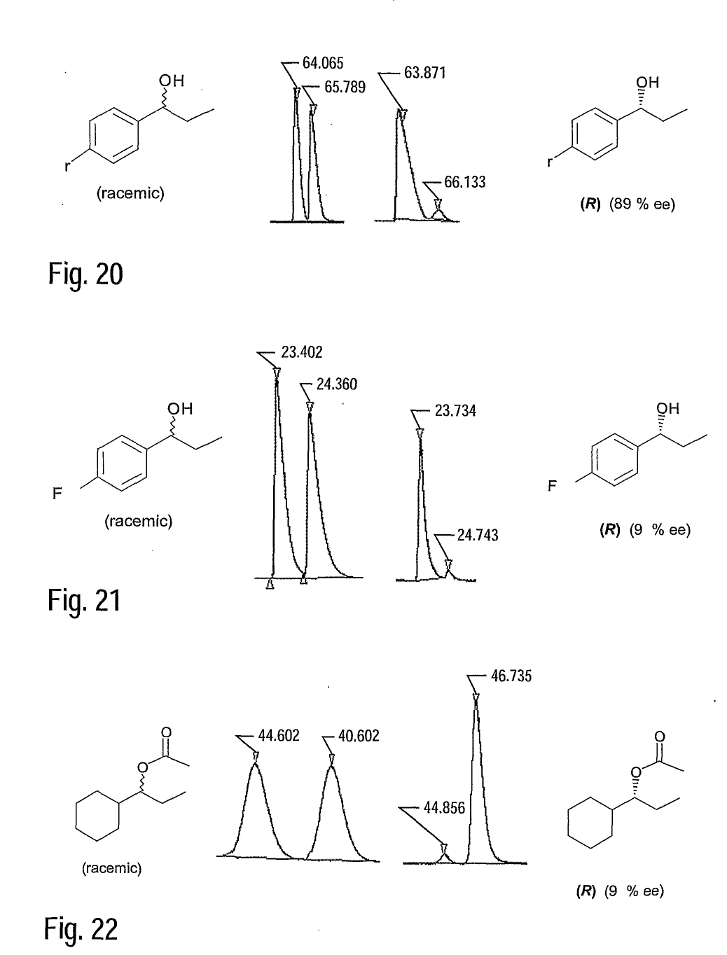

(+)—(R)-4-BrPhCH(Et)OH PA2b. ¹H NMR (500.1 MHz CDCl₃) δ=0.94 (t, J=7.4 Hz, 3 H; CH₂CH₃), 1.75 (ddq, J=14.6, 6.7, 7.4 Hz, 1 H, CH_{2α}CH₃), 1.82 (ddq, J=14.6, 6.6, 7.4 Hz, 1 H, CH_{2β}CH₃), 2.62 (d, J=3.1 Hz, 1 H; OH), 4.59 (dt J=3.1, 6.6, 1 H; CHOH), 7.04-7.11 (m, 2 H; Ar), 7.31-7.36 (m, 2 H; Ar). GC(Cyclodex-B, initially 120° C. ramped to 180° C. at 1° C. min⁻¹): (R)-PA2b (64 min), (S)-PA2b (66 min). GC traces of genuine racemic and chiral catalysis derived PA2b samples are shown in FIG. 20.

(+)—(R)-4-(CF₃)PhCH(Et)OH PA2h. ¹H NMR (500.1 MHz CDCl₃) δ=0.92 (t, J=7.4 Hz, 3 H; CH₂CH₃), 1.75-1.83 (m, 2 H, CH₂CH₃), 2.23 (s, br, 1 H; OH), 4.59 (t, J=6.5, 1 H; CHOH), 7.44 (AB-doublet, J=8.1 Hz, 2 H; Ar), 7.60 (AB-doublet, J=8.1 Hz, 2 H; Ar).

GC(Cyclodex-B, initially 120° C. ramped to 180° C. at 1° C. min⁻¹): (R)-PA2h (23.5 min), (S)-Pa2h (24.5 min). GC traces of genuine racemic and chiral catalysis derived PA20h samples are shown in FIG. 21.

(+)—(R)-c-C₆H₁₁CH(Et)OH PA3m. ¹H NMR (500.1 MHz CDCl₃) δ=0.96 (t, J=7.4 Hz, 3 H, CH₃), 1.21-1.60 (m, 8 H; ring-CH₂ and CH₂CH₃), 1.62-1.82 (m, 4 H; ring-CH₂), 2.19 (s, br, 1 H; OH), 3.27-3.32 (m, 1 H; CHOH). GC(2,6-me-3-pe-γ-CD, 60° C. initially for 5 min, then ramped to 90° C. at 5° C. min⁻¹ using acetate of PA3m): (S)-PA3m-OAc (12.8 min), (R)-PA3m-OAc (13.4 min). GC traces of genuine racemic and chiral catalysis derived PA3m samples (acetates) are shown in FIG. 22.

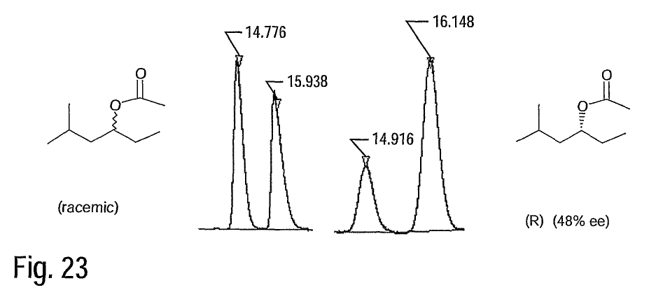

(−)—(R)-i-BuCH(Et)OH PA2n. ¹H NMR (500.1 MHz, CDCl₃) δ=0.91 (d J=6.7 Hz, 1 H; CHCH_{3α}), 0.92 (d J=6.7 Hz, 1 H; CHCH_{3β}) overlapped by 0.93 (t, J=7.4 Hz, 3 H; CH₂CH₃), 1.24 (ddd, J=13.0, 8.8, 4.0 Hz, 1 H; CH_{2α}-i-Pr), 1.32-1.53 (m, 3 H; CH₂CH₃ and CH_{2β}-i-Pr) overlapped by 1.50 (s, br, 1 H. OH), 1.73-1.81 (m, 1 H; CH(CH₃)₂), 3.58-3.63 (m, 1 H; CHOH). GC(6-me-2,3-pe-γ-CD, 50° C. isothermal using acetate of PA2n): (S)-PA2n-OAc (14.8 min), (R)-PA2n-OAc (16.0 min). GC traces of genuine racemic and chiral catalysis derived PA2n samples (acetates) are shown in FIG. 23.

TABLE 1
Alkylation of aldehydes (R¹CHO) with DABAL-(R³)₃ (1) or Al(R³)₃
(R³ = Me, Et).

| | R¹ in RCHO | Method and | | Product (PA) |
|---|---|---|---|---|
| Run | (SC) | Time/h | Yield^{a}/% | Ee (antipode)^{b}/% |
| 1 | Ph (a) | A (Me), 1 | 92 | 91 (R)-(+)-PA1a |
| 2 | Ph (a) | B (Me), 3 | 60 | 85 (R)-(+)-PA1a |
| 3 | Ph (a) | A (Et), 5 | 95 | 86 (R)-(+)-PA2a |
| 4 | Ph (a) | B (Et), 5 | 95 | 83 (R)-(+)-PA2a |
| 5 | 4-BrC₆H₄ (b) | A (Me), 1 | 98 | 93 (R)-(+)-PA1b |
| 6 | 4-BrC₆H₄ (b) | A (Et), 1 | 91 | 89 (R)-(+)-30d |
| 7 | 4-ClC₆H₄ (c) | A (Me), 1 | 79 | 93 (R)-(+)-PA1c |
| 8 | 4-FC₆H₄ (d) | A (Me), 3 | 84 | 94 (R)-(+)-PA1d |
| 9 | 3-FC₆H₄ (e) | A (Me), 1 | 77 | 94 (R)-(+)-PA1e |
| 10 | 2-FC₆H₄ (f) | A (Me), 1 | 81 | 87 (R)-(+)-PA1f |
| 11 | 4-(CN)C₆H₄ (g) | A (Me), 1 | 69 | 94 (R)-(+)-PA1g |
| 12 | 4-(CF₃)C₆H₄ (h) | A (Me), 1 | 58 | 94 (R)-(+)-PA1h |
| 13 | 4-(CF₃)C₆H₄ (h) | A (Et), 1 | 90 | 95 (R)-(+)-30h |
| 14 | 3-(CF₃)C₆H₄ (i) | A (Me), 1 | 93 | 93 (R)-(+)-PA1i |
| 15 | 4-MeC₆H₄ (j) | A (Me), 3 | 90 | 81 (R)-(+)-PA1j |
| 16 | 3-MeC₆H₄ (k) | A (Me), 1 | 82 | 93 (R)-(+)-PA1k |
| 17 | 2-MeC₆H₄ (l) | A (Me), 1 | 78 | 90 (R)-(+)-PA1l |
| 18 | c-C₆H₁₁^{c} (m) | A (Me), 1 | 80 | 94 (R)-(−)-PA1m |
| 19 | c-C₆H₁₁^{c} (m) | A (Et), 10 | 60^{d} | 91 (R)-(+)-PA3m |
| 20 | c-C₆H₁₁^{c} (m) | B (Et), 1.5 | 55 | 7 (R)-(+)-PA3m |
| 21 | i-Bu (n) | B (Me), 6 | 60 | 95 (R)-(−)-PA1n |
| 22 | i-Bu (n) | B (Et), 6 | 72 | 48 (R)-(−)-PA2n |
| 23 | t-BuCH₂ (o) | B (Me), 7 | 95 | 67 (R)-(−)-PA1o |
| 24 | n-C₆H₁₃ (p) | B (Me), 6 | 91 | 61 (R)-(−)-PA1p |
| 25 | PhCH₂CH₂ (q) | B (Me), 6 | 94 | 77 (R)-(−)-PA1q |
| 26 | (E)-PhCH═CH (r) | A (Me), 1 | 94 | 66 (R)-(+)-PA1r |

^{a}GC or isolated: GC vs. internal standard on 0.25 mmol (RCHO) scale reaction; isolated yields were within 2 to 5% of these figures on 1.0 mmol scale duplicate reactions. Compounds 4 and 5 were authenticated against genuine samples prepared either by achiral Ph₃P (2 mol %)/Ni(acac)₂ (1 mol %) catalysed reactions using 1 and/or NaBH₄ reduction of the parent ketones.
^{b}Determined by GC on a Cyclodex-B column in all cases except PA1a, PA2a (GC on Lipodex-A), PA1q-r (GC on 2,6-me-3-pe-γ-CD) and PA1m-p, PA3m-n (which were by GC on the acetate of isolated sec-alcohol as described before). Stereo correlation based on the sign of optical rotation or by GC comparison with authentic samples of known configuration.
^{c}1.0 Eq. of DABAL-R₃ 1 used.
^{d}Mass balance accounted for by α-deprotonation derived by-products.

TABLE 2
Alkylation of (E)-cinamaldehyde with DABAL-(Me)₃ Showing
exemplifying ligand family used.

| | | Method and | | Product |
|---|---|---|---|---|
| Run | Ligand Used | Time/h | Yield^{a}/% | Ee (antipode)^{b}/% |
| 26 | L1^{c} | A (Me), 1 | 94 | 62 (R)-(+)-PA1r |
| 2 | L2 | A (Me), 1 | 71 | 66 (S)-(−)-PA1r |
| 2 | L3 | A (Me), 1 | (81) | 59 (R)-(+)-PA1r |
| 2 | L4 | A (Me), 1 | (76) | 38 (R)-(+)-PA1r |
| 2 | L5 | A (Me), 1 | 50 | 33 (R)-(+)-PA1r |
| 2 | L6 | A (Me), 1 | 60 | 80 (R)-(+)-PA1r |

^{a}Determined by GC against internal standard as in Table 1 or by isolation
(shown in parentheses).
^{b}Determined by chiral GC as in Table 1.
^{c}Typical ligand structures are shown below

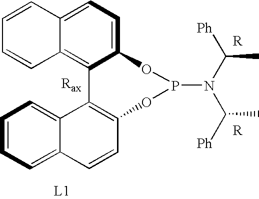

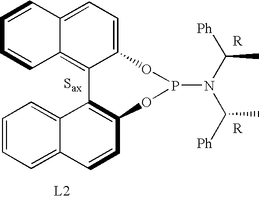

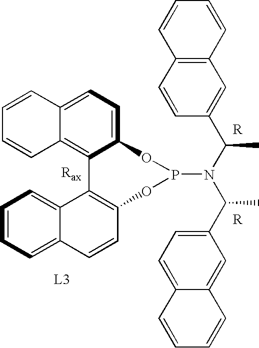

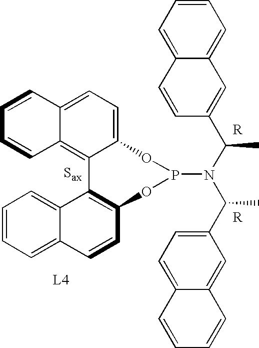

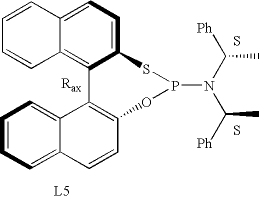

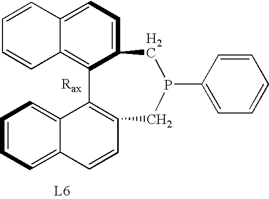

Various alternatives and modifications thereof can be devised by those skilled in the art without departing from the spirit and scope of the present invention. Accordingly, the present invention is intended to embrace all such alternatives, modifications, and variations that fall within the scope of the appended claims. The present invention has been described with particular reference to the preferred embodiments. It should be understood that the foregoing descriptions and examples are only illustrative of the invention.

1. A process for converting a carbonyl group within a substrate to a chiral alcohol moiety comprising reacting the carbonyl containing substrate with an organoaluminium reagent in the presence of a Group 5-12 transition metal based catalyst which is complexed with a chiral ligand.

2. A process as claimed in claim 1 wherein the carbonyl containing substrate is of formula SC
R¹R²CO
(SC)
and the chiral alcohol is of formula PA
R¹R²R³COH
(PA)
wherein R¹, R² and R³ are each independently C₁₋₂₄ alkyl, alkenyl, allyl, alkynyl, aryl, or heteroaryl each of which may be substituted, wherein one of R¹ and R² is H.

3. A process according to claim 1 with the proviso that when the carbonyl containing compound is an aldehyde, the transition metal is Ni and the organometallic is AlR³R⁴R⁵ or is complexed with a polyamine 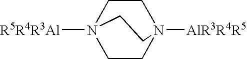
where R³, R⁴ and R⁵ are all methyl or are all ethyl, the chiral ligand is not L1

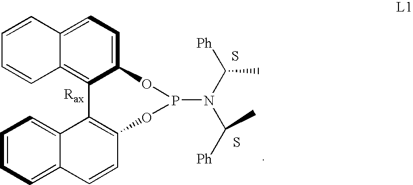

4. A process according to claim 1 wherein the organoaluminium reagent is complexed with an amine or polyamine (hereinafter (poly)amine) capable of acting as a Lewis base in binding to the organoaluminium reagent.

5. A process as claimed in claim 4 wherein an organoaluminium—(poly)amine complex comprises an aluminum to (poly)amine as defined in a ratio of 1:1 to 1:4, preferably 1: 1, 1:2 or 1:4, most preferably 1:2.

6. A process according to claim 1 wherein the organoaluminium reagent has the general structure AlR³R⁴R⁵ wherein each R³ to R⁵ is independently C₁₋₂₄ alkyl, alkenyl, alkynyl, alkoxy, aminoalkyl, aryl, heteroaryl, allyl or substituted versions thereof, wherein substituents are alkyl, alkoxy, amino, alkenyl, alkynyl, allyl, or one or two of R⁴ and R⁵ are halide or pseudohalide, or R⁵ is H, and wherein when R⁵ is H, the organoaluminium reagent may be associated with one or two additional molecules of AlR³R⁴R⁵, R⁵ being H, in dimerised form.

7. A process according to claim 6 wherein the halide is chlorine or bromine.

8. A process according to claim 6 where the organoaluminium—polyamine complex is of the formula 1a 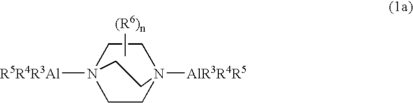
wherein each R³ to R⁵ are as hereinbefore defined and one or more R⁶ are as hereinbefore defined for R³ and n is zero or is a whole number integer selected from 1 to 12.

9. A process according to claim 6 where the organoaluminium—polyamine complex is of the formula 1b 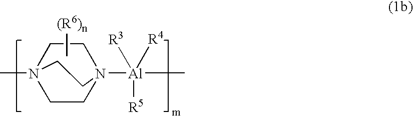
wherein each R³, R⁴, R⁵ is as hereinbefore defined and one or more R⁶ are as hereinbefore defined for R³ and n is zero or is a whole number integer selected from 1 to 12 and m is a whole number integer selected from 2 to 300.

10. A process of claim 3 wherein R³ and R⁴ are C₁₋₅ alkyl, C₃₋₁₄ alkenyl, alkynyl, allyl, aryl, preferably C₁₋₅ alkyl, C₃₋₁₄ alkenyl, and R⁵ is H, C₁₋₅ alkyl, C₃₋₁₄ alkenyl, alkynyl, allyl, aryl and halide, preferably H, C₃₋₁₄ alkenyl, alkynyl and allyl.

11. A process of claim 4 wherein in an organoaluminium reagent of formula 1, 1a, or 1b, R³ is different to and of different reactivity to at least one of R⁴ and R⁵ such as to induce addition of R³ to the carbonyl containing substrate.

12. A process according to claim 4 wherein the organoaluminium—polyamine complex is of the formula 2 to 5 or 14 to 17 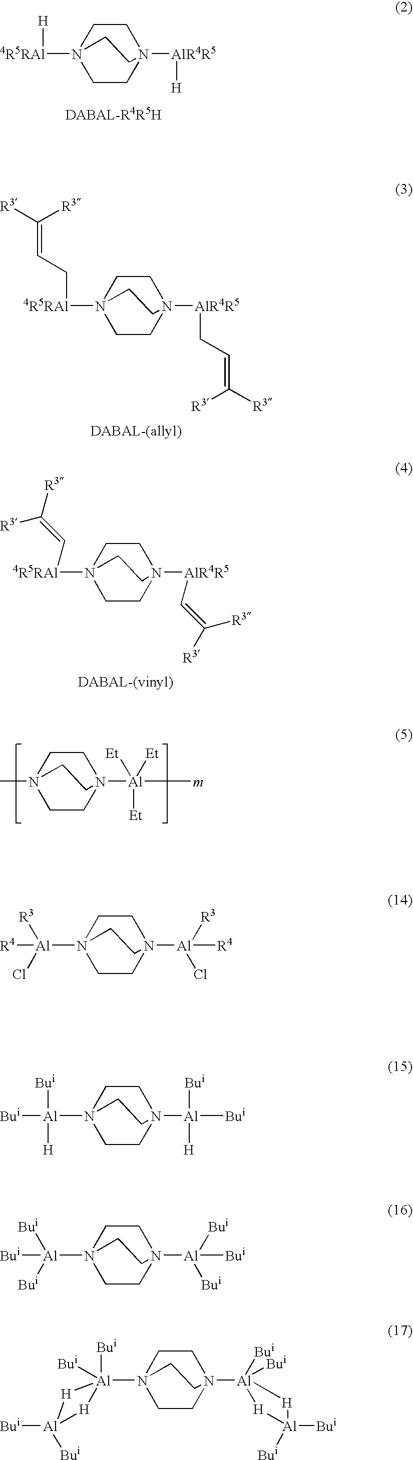
wherein each R³′ or R³″ independently, where shown, is as hereinbefore defined for is as hereinbefore defined for R³ or one of R³′ and R³″ is H, and m is from 2 to 50.

13. A process according to claim 4 in which the organoaluminium—polyamine complex is a solid and is used in coated, pelletized or tabletised form, wherein a coating is selected from a binding additive such as polyacrylate.

14. A process of claim 1 wherein the chiral ligand is of the formula 26 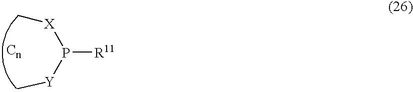
wherein Cₙ together with the X, Y and P atoms forms a ring with 2-4 C atoms which may be substituted or unsubstituted or form a fused or spiro mono or polycyclic aromatic structure, and together with any substitutents or fused or spiro mono or polycyclic aromatic structure comprises 6-45 C atoms and heteroatoms; X and Y are each independently selected from O, S, CH₂, CNH, CR²⁰₂, CR²⁰H where R²⁰ is selected from alkyl, allyl, vinyl, aryl, heteroaryl or substituted variants thereof, wherein substituents are selected from aryl, alkyl, alkoxy, hydroxy, nitrile, halogen or carbonyl and the like, preferably Ph, Ar, OMe, OCOR⁷ etc or R¹¹ is part of a polymeric backbone and X—Cₙ—Y is any aliphatic or aromatic tether that engenders the phosphorus to become chiral at that centre and R¹¹ is selected from H, OH or substituted or unsubstituted C₁₋₂₄ alkyl, alkenyl, aryl, aralkyl, alkaryl, heteroaryl or heterocyclic, or OR⁷, SR⁷, NHR⁷, NR⁷₂ where R⁷ is as defined for R¹¹ or is any linear, cyclic or branched alkyl or aryl capable of bearing additional substituents selected from aryl, alkyl, alkoxy, hydroxy, nitrile, halogen or carbonyl and the like, preferably Ph, Ar, OMe OCOR⁷ etc.

15. A process according to claim 1 wherein the chiral ligand is of the formula 22 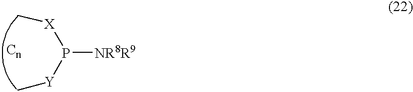
where R⁸ and R⁹ are as defined for R¹¹ or R⁸ and R⁹ form a heterocyclic or heteroaryl ring with the N atom to which they are bound, and are preferably substituted or unsubstituted aryl groups, heteroaryl groups, aliphatic groups or combinations thereof.

16. A process according to claim 1 wherein the chiral ligand is of the formula 22a or 26a 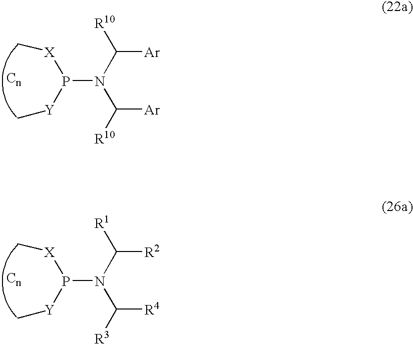
wherein Ar is a substituted or unsubstituted aryl or heteroaryl and Cₙ, X and Y are as hereinbefore defined, and R¹⁰ is as defined for R¹¹ and is preferably alkyl more preferably methyl.

17. A process according to claim 1 wherein the chiral ligand is of the formula 8 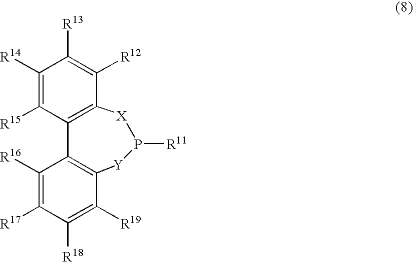
wherein X and Y are each independently selected from O, S, CH₂, NH, NR²⁰, CR²⁰₂, CR²⁰OH where R²⁰ is selected from alkyl, allyl, vinyl, aryl, heteroaryl or substituted variants thereof and R¹¹ is as hereinbefore defined, and each R¹²⁻¹⁹is independently selected from H, R¹¹ as hereinbefore defined, COalkyl, COaryl, OCOAlkyl, OCOAryl, F. Cl, Br, OH, NO₂, Trialkylsilyl, CF₃, CN, CO₂H, CHO, Salkyl, Saryl, SOalkyl, SOaryl, SO₂alkyl, SO₂aryl, SO₃H, SO₃alkyl, SO₃aryl, CO₂NH₂, CONH₂, CONHalkyl, CONH(alkyl)₂, NHCOH, NHCOalkyl, CH═CHCO₂alkyl, CH—CHCO₂H etc and preferably one of R¹² to R¹⁵ and R¹⁶ to R¹⁹ is any aryl or heteroaryl group, or wherein any two adjacent groups R¹² to R¹⁹, preferably R¹² and R¹³, R¹³ and R¹⁴, R¹⁴ and R¹⁵ or R¹⁶ and R¹⁷ or R¹⁷ and R¹⁸ or R¹⁸ and R¹⁹ or R¹⁵ and R¹⁶, together with the X—Cₙ—Y structure, form one or more single or fused or spiro aromatic ring structures,
and at least one R¹² is not H and is such as to form a chiral centre, or R¹¹ is substituted, preferably is NR⁷₂ or where R⁷ is C₁₋₅alkyl or aryl and is not H, such as to form a chiral centre.

18. A process according to claim 1 where the chiral ligand is of formula 8a 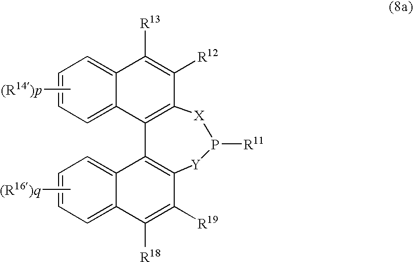
wherein X, Y and R¹¹ and R¹², R¹³, R¹⁸and R¹⁹ are as hereinbefore defined, p and q are each independently zero or a whole number integer from 1 to 4.

19. A process according to claim 1 wherein preferably alkyl and alkenyl comprise 1 to 4 C atoms, whereby an alkenyl group comprises one double bond, and aryl comprises 5 to 7 C atoms, heteroaryl groups comprise preferably one or two N atoms, a N and an O atom or a S and an O atom.

20. A process according to claim 3 wherein R¹¹ is optionally substituted as hereinbefore defined and wherein substituents include OR¹¹, SR¹¹, Ph, aryl, alkyl, alkenyl, heteroaryl, NHR⁹, NR⁹R¹⁰ where R⁹ is CR⁶R⁷ and R⁶ and R⁷ are each independently selected from methyl, phenyl, naphthyl, preferably methyl and phenyl respectively.

21. A process according to claim 2 wherein the chiral ligand is selected from formulae 6, 7, 8, 9, 10 or 11 or 12

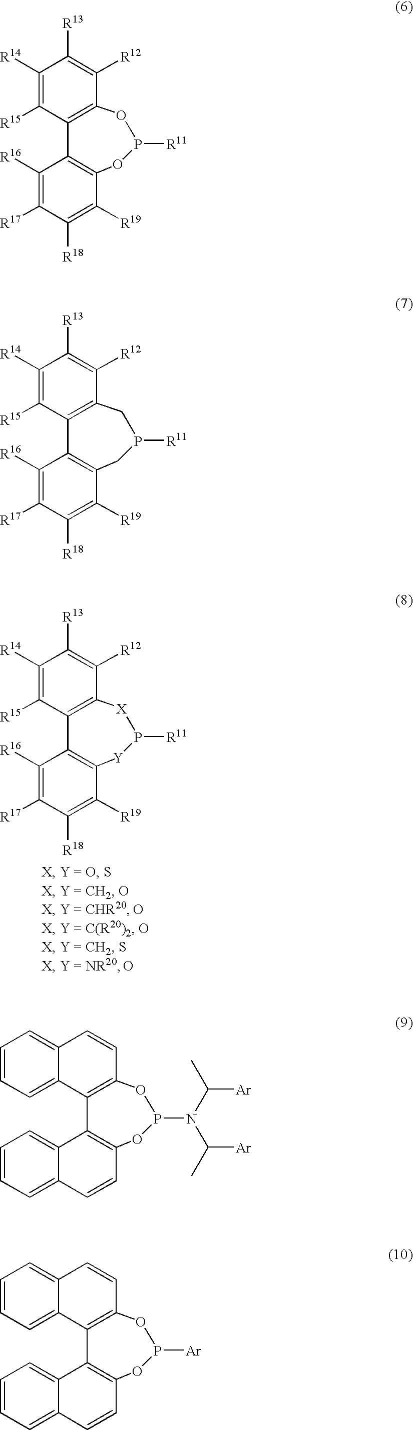

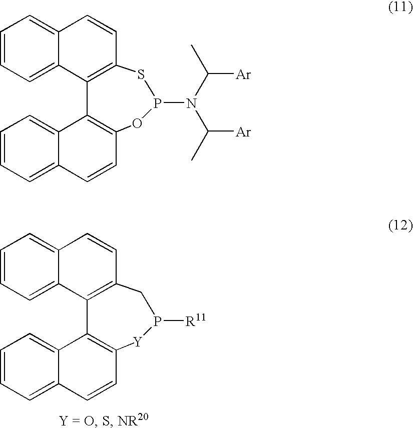

22. A process according to claim 2 wherein the chiral ligand is selected from formulae L1-L6 or 23 or L7-L9

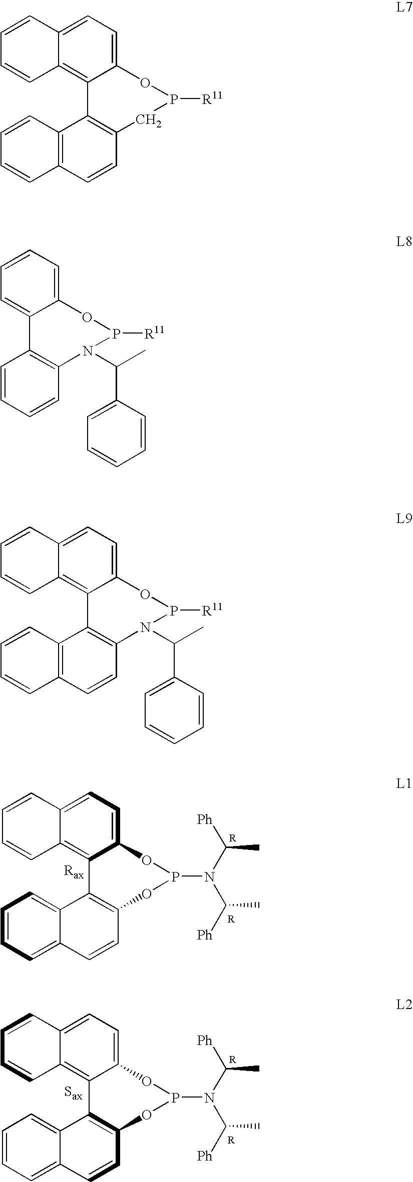

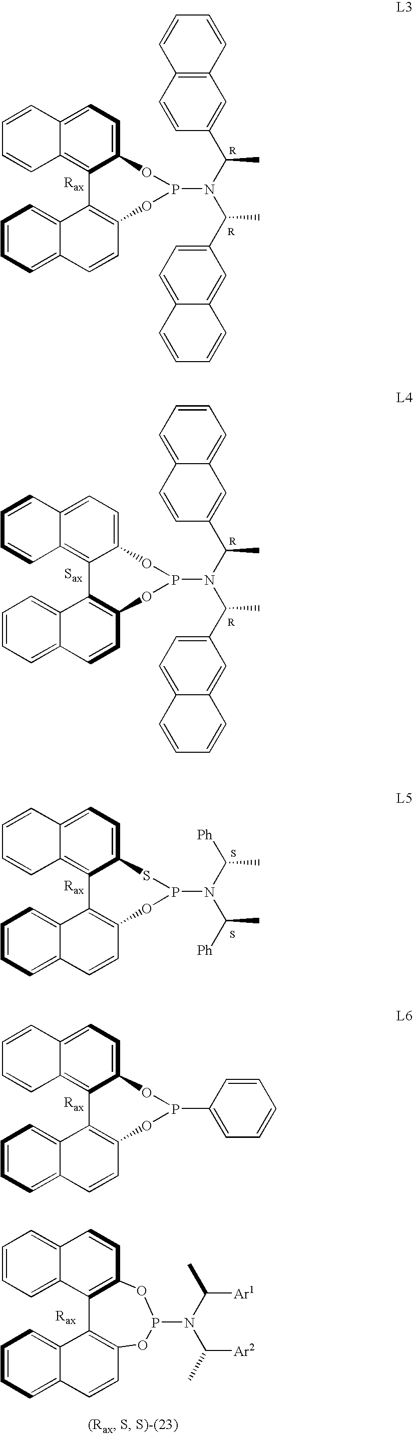

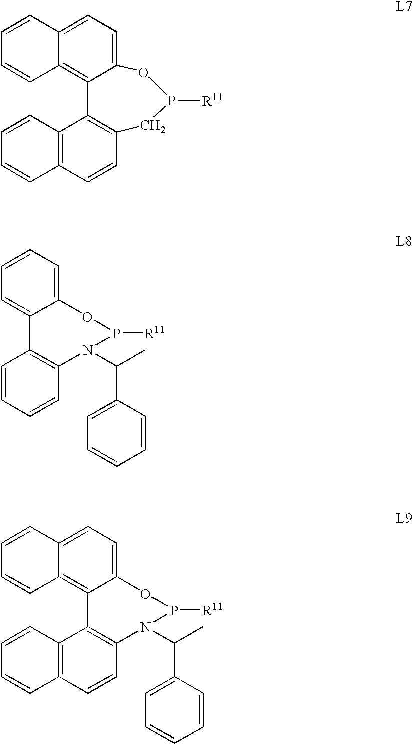

23. A process according to claim 2 wherein X and Y are respectively O and S; or O and CH₂, CHR²⁰ or CR²⁰₂; or O and NH; or O and NR²⁰; or S and CH₂, CHR²⁰ or CR²⁰₂; or are both O; or are both CH₂, CHR²⁰ or CR²⁰₂; preferably are O and CH₂, CHR²⁰ or CR²⁰₂; or O and S; or O and NH or NR²⁰.

24. A process according to claim 1 wherein the Group 5-12 transition metal based catalyst is complexed with a chiral ligand ex situ or in situ

25. A process according to claim 1 where the Group 5-12 transition metal is nickel, preferably Ni(acac)₂.

26. A process according to claim 1 where the carbonyl group is present in a prochiral aldehyde and the product of the reaction is a chiral secondary alcohol or a mixture of the two enantiomeric forms of a chiral secondary alcohol in any proportion.

27. A process according to claim 1 where a compound that contains a carbonyl group is R¹C(═O)R where R¹ and R² are each independently selected from linear, cyclic or branched alkyl, alkenyl, alkynyl, allyl, aromatic or heteroaromatic and can contain one or more functional groups or substituents selected from CO₂R, COR, CONH₂ SO₃R, OH, OR, NH₂, NHR, Cl, Br, I, NO₂, alkenyl, allyl, alkynyl, aryl, heteroaryl, and the like, wherein substituents as side chains may bear functional groups as defined, or R² is H.

28. A process according to claim 1 wherein the solvent is ethereal such as tetrahydrofuran and uses any reaction element of the preceding claims.

29. An organoaluminium compound complexed with a polyamine, of the formula 1a 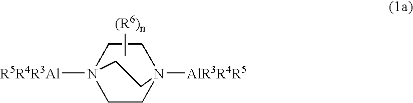
wherein each R³ to R⁵ is independently selected from C ₁₋₂₄ alkyl, alkenyl, alkynyl, alkoxy, aminoalkyl, aryl, heteroaryl, allyl and substituted versions thereof, wherein substituents are selected from alkyl, alkoxy, amino, alkenyl, alkynyl, allyl, or one or two of R⁴ and R⁵ are halide or pseudohalide, or R⁵ is H, and wherein when R⁵ is H, the organoaluminium reagent may be associated with one or two additional molecules of AlR³R⁴R⁵, R⁵ being H, in dimerised form and one or more R⁶ are as hereinbefore defined for R³ and n is zero or is a whole number integer selected from 1 to 12 with the proviso that when n is 0, each of R³,R⁴ and R⁵ are not simultaneously CH₃; or as herein disclosed in the description, claims, examples and/or figures.

30. An organoaluminium compound complexed with a polyamine, of the formula 1b 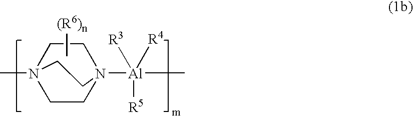
wherein each R³, R⁴, R⁵is independently selected from C₁₋₂₄ alkyl, alkenyl, alkynyl, alkoxy, aminoalkyl, aryl, heteroaryl, allyl and substituted versions thereof, wherein substituents are selected from alkyl, alkoxy, amino, alkenyl, alkynyl, allyl, or one or two of R⁴ and R⁵ are halide or pseudohalide, or R⁵ is H, and wherein when R⁵ is H, the organoalumninium reagent may be associated with one or two additional molecules of AlR³R⁴R⁵, R⁵ being H, in dimerised form and one or more R⁶ are as hereinbefore defined for R³ and n is zero or is a whole number integer selected from 1 to 12 and wherein m is a whole number integer selected from 2 to 300.

31. An organoaluminium compound complexed with a polyamine according to claim 29 wherein R³ and R⁴ are selected from C₁₋₅ alkyl, C₃₋₁₄ alkenyl, alkynyl, allyl, aryl, preferably C₁₋₅ alkyl, C₃₋₁₄ alkenyl, and R⁵ is selected from H, C₁₋₅ alkyl, C₃₋₁₄ alkenyl, alkynyl, allyl, aryl and halide, preferably H, C₃₋₁₄ alkenyl, alkynyl and allyl.

32. An organoaluminium compound complexed with a polyamine according to claim 29 wherein in an organoaluminium reagent of formula 1 or 1a or 1b , R³ is different to and of different reactivity to at least one of R⁴ and R⁵ such as to induce addition of R³ to the carbonyl containing substrate.

33. An organoaluminium—polyamine complex of the formula 2 to 5 or 14 to 17 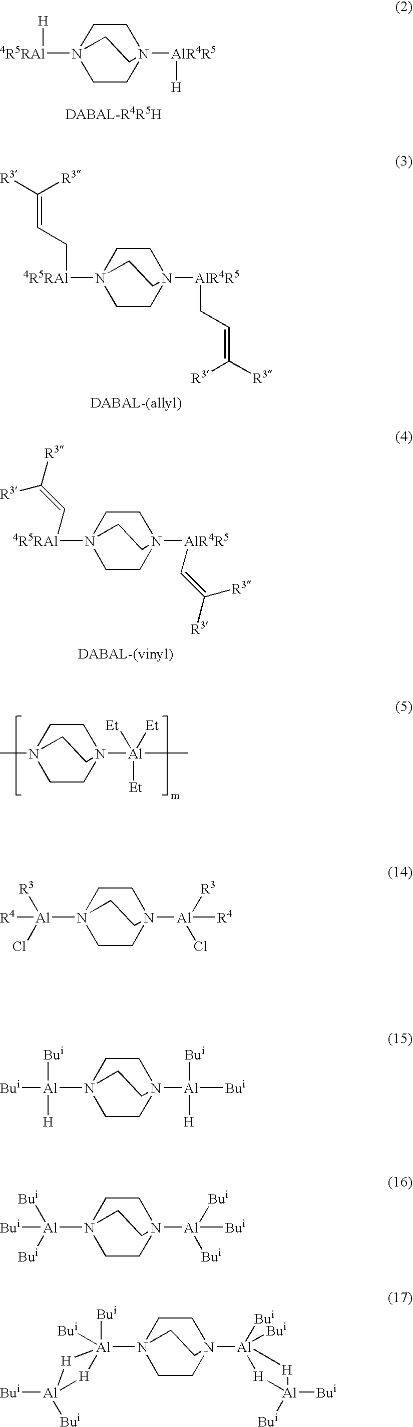
wherein each R³′ or R³″ independently, where shown, is as hereinbefore defined for R³ or one of R³′ and R³″ is H, and m is from 2 to 50, and R⁴ R⁵ are as defined herebeforein.

34. An organoaluminium—polyamine complex according to claim 29 which is a solid and is in coated, pelletized or tabletised form, wherein a coating is selected from a binding additive such as polyacrylate.

35. A chiral ligand of formula 26 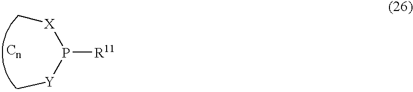
according to claim 16 with the proviso that when X is O and Y is O, R¹¹ is not NR⁸R⁹ wherein R⁸ and R⁹ are H, optionally substituted alkyl, aryl, aralky, alkaryl or form a hetero ring with the N atom (I), or when the ligand is of formula 8 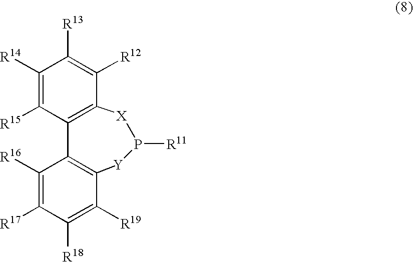
as hereinbefore defined, and one of X and Y is CH₂, the other of X and Y is not CH₂ (ie formula II below), or when the ligand is of formula 26 or 8 or when Cₙ is unsubstituted naphthyl, Y is O, X is NH, R¹¹ is not alkyl, aryl, arylene, substituted aryl, heteroaryl, phenol, ferrocene, or aryl carboxylate and in particular phenyl (ie formula III below)

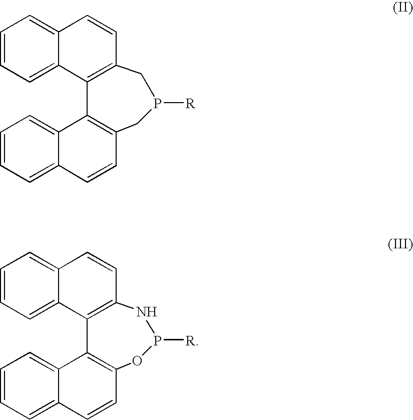

36. A catalytic composition comprising an organoaluminium compound complexed with a polyamine as hereinbefore defined in claim 29.

37. A catalytic composition comprising a chiral ligand as hereinbefore defined in claim 35.

38. A catalyst composition according to claim 37 in which the chiral ligand is complexed with a transition metal containing compound.

39. A catalyst composition comprising the chiral ligand of formula 26 or according to claim 14 complexed with a transition metal containing compound, preferably is nickel(I), with the proviso that when X and Y are both O and R¹¹ is NR⁸R⁹ as hereinbefore defined the transition metal is not rhodium or ruthenium, or with the proviso that when the ligand is of formula 8 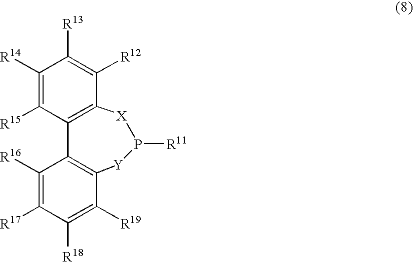
as hereinbefore defined, and one of X and Y is CH₂, the other of X and Y is not CH₂ (i.e. formula II as defined in claim 35), or when the ligand is of formula 26 or 8 or when Cₙ is unsubstituted naphthyl, Y is O, X is NH, R¹¹ is not alkyl, aryl, arylene, substituted aryl, heteroaryl, phenol, ferrocene, or aryl carboxylate and in particular phenyl (ie formula III as defined in claim 35).

40. A catalytic composition according to claim 38 in which the chiral ligand is complexed with a nickel containing compound.

41. (canceled)

42. (canceled)

43. A chiral alcohol moiety obtained with the process or entity of claim 1.

44. (canceled)

45. A process for the preparation of a chiral ligand as hereinbefore defined in claim 1 comprising reaction of a compound of formula 30

| | |
|---|---|
| | (30) |
| 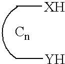 | |
| X | Y |
|---|---|
| O | S |
| O | CH₂ |
| O | CHR₂²⁰ |
| O | CR₂²⁰ |
| O | NR²⁰ |

and (LG)₂PR¹¹ or (LG)₂PN(CHR²¹R²²)(CHR²³R²⁴) reagents (LG=a suitable leaving group, such as Cl, Br, I, OEt, OMe, OPh, OAr, OR) and in the presence of a base or nBuLi/TMEDA.

46. A process for preparing an intermediate of formula 30 as claimed in claim 45 comprising the reaction of a disulfonyl precursor 31 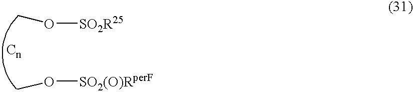
where R^{perF} is any perfluorinated C₁₋₅ alkyl, preferably CF₃ and R²⁵ is alkyl, alkenyl, aryl for example phenyl, 4-tolyl, tBu or CH₃
to form the intermediate wherein X and Y are O and CH₂ CHR²⁰, CR²⁰, with a source of organometallic anions, such as R³R⁴CHMgBr preferably R³ and or R⁴=H, Me, in the presence of a nickel catalyst with subsequent hydrolysis to OH, e.g. with KOH;
or to form the intermediate wherein X and Y are O and NR²⁰, with H₂NR²⁰ and Pd₂(dba)₃, Xantphos (9,9-Dimethyl-4,5-bis(diphenylphosphino)xanthene, Chemical Abstracts No. [161265-03-8]) and LiHDMS.

47. A process for preparing an intermediate of formula 31 as claimed in claim 46 comprising the reaction of a diol precursor 32 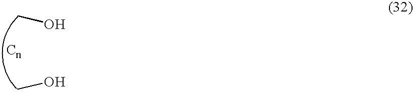
with R²⁵SO₂Cl followed by R^{perF}SO₂(LG) where LG is any viable leaving group (e.g. F, Cl, OSO₂R^{perF}, etc.).

48. A novel intermediate of formula 30 and 31 with the proviso that X,Y is not S, O or CH₂,O.

49. A novel intermediate of formula 33a, 33b, 34a, 34b, 35, 36b, 36c, 37 or intermediate (LG)₂PN(CHR²¹R²²)(CHR²³R²⁴). [chemical structure]
